(19) United States
(12) Patent Application Publication
(10) Pub. No.: US 2023/0058380 A1
(43) Pub. Date: Feb. 23, 2023

(54) INTRAPANCREATIC M2 POLARIZATION OF MACROPHAGES TO TREAT TYPE 1 DIABETES

(71) Applicant: University of Pittsburgh - Of the Commonwealth System of Higher Education, Pittsburgh, PA (US)

(72) Inventors: George Gittes, Pittsburgh, PA (US); Xiangwei Xiao, Pittsburgh, PA (US)

(73) Assignee: University of Pittsburgh - Of the Commonwealth System of Higher Education, Pittsburgh, PA (US)

(21) Appl. No.: 17/789,739

(22) PCT Filed: Dec. 29, 2020
(86) PCT No.: PCT/US2020/067281

§ 371 Date: Jun. 28, 2022

Related U.S. Application Data
(60) Provisional application No. 62/955,322, filed on Dec. 30, 2019.

(51) Int. Cl. A61K 48/00; A61P 3/10

(52) U.S. Cl. CPC A61K 48/0058; A61K 48/0075; A61P 3/10

(57) ABSTRACT

Methods are disclosed for polarizing macrophages to become M2 macrophages. Methods also are disclosed for treating type 1 diabetes in a subject. These methods include administering to the subject a vector comprising a macrophage specific promoter operably linked to a nucleic acid molecule encoding TNF-alpha-induced protein 8-like 2 (TIPE2) protein. In some embodiments, the vector is administered locally to a pancreas of the subject. In further embodiments, compositions are disclosed including a) a vector comprising a macrophage specific promoter operably linked to a nucleic acid molecule encoding TNF-alpha-induced protein 8-like 2 (TIPE2) protein; b) a buffer; and c) a contrast dye for endoscopic retrograde cholangiopancreatography.

### CROSS REFERENCE TO RELATED APPLICATIONS

This claims the benefit of U.S. Provisional Application No. 62/955,322, filed Dec. 30, 2019, which is incorporated herein by reference.

### STATEMENT OF GOVERNMENT SUPPORT

This invention was made with government support under DK112836 awarded by the National Institutes of Health. The government has certain rights in the invention.

### FIELD OF THE DISCLOSURE

This relates to the field of diabetes, specifically to the intraductal administration of a viral vector encoding TNF-alpha-induced protein 8-like 2 (TIPE2), to induce the induction of M2 macrophage polarization and treat diabetes.

### BACKGROUND

Insulin, produced by the pancreatic beta cells, is a key regulator of glucose homeostasis. Insufficient insulin leads to diabetes, a metabolic disease that affects over 300 million people worldwide (Bluestone, et al., Nature 464, 1293-1300 (2010)). Type 1 diabetes (T1D) is usually diagnosed in children and young adults, constituting about 5% of all diabetes. Compared to patients with type 2 diabetes (T2D), T1D patients have more severe symptoms and complications, and experience an earlier onset and rapid progression of disease (Bluestone, et al., Nature 464, 1293-1300 (2010)). T1D is characterized by a pathogenic, significantly reduced beta-cell mass, resulting from the autoimmune destruction of pancreatic beta-cells mediated mainly by T-cells and macrophages (Pipeleers et al., Diabetes Obes Metab 10 Suppl 4, 54-62 (2008); Mathis, et al., Nature 414, 792-798 (2001).

To successfully cure T1D, both regeneration of functional beta-cells and suppression of autoimmunity seem necessary. So far, a clinically applicable approach to meet these goals is lacking. Pancreatic duct infusion of an adeno-associated virus (AAV) carrying Pdx1 and MafA expression cassettes (AAV-PM) can reprogram alpha-cells into functional beta-like cells and can normalize blood glucose in autoimmune non-obese diabetes (NOD) mice (the widely accepted mouse model for T1D) for approximately 4 months (Xiao et al., Cell Stem Cell 22, 78-90 e74 (2018)). Recurrence of diabetes in these mice likely results from recurrent autoimmunity, with the eventual recognition of the newly formed beta-cells by autoimmunity. Thus, to successfully cure T1D, effective suppression of autoimmunity may be required, in addition to generating new beta-cells. A need remains for methods for suppressing autoimmunity in subject with T1D, thereby providing treatment.

### SUMMARY OF THE DISCLOSURE

Methods are disclosed for polarizing macrophages to become M2 macrophages. These methods include administering to the subject a vector comprising a macrophage specific promoter operably linked to a nucleic acid molecule encoding TNF-alpha-induced protein 8-like 2 (TIPE2). In some embodiments, the vector is administered locally to a pancreas of the subject.

In some embodiments, methods are disclosed for treating T1D in a subject. These methods include administering to the subject a vector comprising a macrophage specific promoter operably linked to a nucleic acid molecule encoding TNF-alpha-induced protein 8-like 2 (TIPE2) protein, wherein the vector is administered locally to a pancreas of the subject, thereby polarizing macrophages to become M2 macrophages and treating the T1D in the subject.

In further embodiments, compositions are disclosed including a) a vector comprising a macrophage specific promoter operably linked to a nucleic acid molecule encoding TNF-alpha-induced protein 8-like 2 (TIPE2) protein; b) a buffer; and c) a contrast dye for endoscopic retrograde cholangiopancreatography.

The foregoing and other features and advantages of the invention will become more apparent from the following detailed description of several embodiments which proceeds with reference to the accompanying figures.

### BRIEF DESCRIPTION OF THE FIGURES

FIGS. 1A-1G. TIPE2 is expressed in M2 macrophages and forced expression in M1 macrophages can trigger M2 polarization. (A) M1 and M2 macrophages in the dissociated 16-week old female NOD mouse pancreas were sorted by flow cytometry, based on F4/80 (all macrophages) and CD206 expression (M1: CD206−; M2: CD206+). The vast majority of the macrophages in the NOD pancreas are M1. (B-C) TIPE2 levels were assessed by RT-qPCR (B), and by Western blotting (C) in M1 and M2 macrophages. (D) Schematic of AAV-pCD68-TIPE2 and AAV-pCD68-GFP vector constructs. (E) Western blotting for TIPE2 in AAV-pCD68-TIPE2 or AAV-pCD68-GFP-transduced M1 macrophages. (F) RT-qPCR for M1/M2 macrophage-associated genes. (G) Representative images for arginine 1 (ARG1) and GFP immunostaining on AAV-pCD68-TIPE2 or AAV-pCD68-GFP-transduced M1 macrophages. FSC: forward scatter. HO: Hoechst nuclear staining. Mϕ macrophages. **p<0.01. *p<0.05. N=5. Scale bars are 50 μm.

FIGS. 2A-2F. TIPE2-induced M2-macrophage polarization in the pancreas reverses new-onset diabetes in NOD mice. (A) In vivo specificity of AAV-pCD68-TIPE2 for macrophages in the NOD mouse pancreas. Representative images for F4/80 and GFP immunostaining on AAV-pCD68-TIPE2-infused NOD mouse pancreas at day 7 shows GFP signal exclusively in F4/80+ macrophages. (B) RT-PCR for GFP in F4/80+ and F4/80− pancreatic cells. Positive control (+) and negative control (−) were a GFP-plasmid and water used as templates, respectively. (C-D) Flow cytometry for CD206+ cells in total F4/80+ cells from AAV-pCD68-TIPE2-infused NOD mouse pancreas, shown by quantification (C) and by representative FACS plots (D). (E-F) Effects of TIPE2-induced, pancreas-specific M2 macrophage polarization on diabetes status in NOD mice, shown by diabetes progression rate (E), and by fasting blood glucose (F). *p<0.05. NS: non-significant. For panel A-D, N=5. For panel E-F, N=10. Mϕ: macrophages. Scale bars are 30 μm.

FIGS. 3A-3H. Role of Foxp3+ Tregs in mediating the reversal of autoimmune diabetes in NOD mice by TIPE2-induced M2-macrophage polarization. (A-B) Flow cytometry for CD8+ cytotoxic T-cells in total pancreatic cells from the AAV-pCD68-TIPE2 or AAV-pCD68-GFP-treated NOD mouse at day 7, shown by representative FACS plots (A) and by quantification (B). (C-D) Flow cytometry for Foxp3+ Tregs in total pancreatic cells from the AAV-pCD68-TIPE2 or AAV-pCD68-GFP-treated NOD mouse pancreas at day 7, shown by representative FACS plots (C) and by quantification (D). (E) Schematic of AAV-pFoxp3-DTA vector and control AAV-pFoxp3-null. (F-G) Flow cytometry for Foxp3+ Tregs in the AAV-pFoxp3-DTA/AAV-pFoxp3-null-treated, AAV-pCD68-TIPE2-ductal infused NOD mouse pancreas at day 14 after viral treatment, shown by representative FACS plots (F) and by quantification (G). (H) Fasting blood glucose. FSC: forward scatter. *p<0.05. N=10.

FIGS. 4A-4F. TIPE2-triggered M2 polarization of tissue-resident macrophages induces upregulation of CRIg to increase Treg numbers. (A-B) Immunostaining for CRIg+ cells in wild-type mouse pancreas, NOD mouse pancreas prior to viral treatment, or NOD mouse pancreas 7 days after ductal infusion with AAV-pCD68-TIPE2 or AAV-pCD68-GFP, shown by representative images (A) and by quantification (B). (C-E) An intraperitoneal (i.p.) injection of neutralizing antibody against CRIg (aCRIg) or control IgG into AAV-pCD68-TIPE2-treated NOD mice twice per week was performed. (C) Fasting blood glucose. (D-E) Flow cytometry for CD8+ cytotoxic T-cells and Foxp3+ Tregs in the aCRIg/IgG-administrated, AAV-pCD68-TIPE2-treated NOD mouse pancreas at day 7, shown by quantification (D) and by representative FACS plots (E). (F) TIPE2-triggered M2 polarization of tissue-resident macrophages induces upregulation of CRIg, which subsequently reverses diabetes progression in the NOD mouse through suppression of effector cytotoxic T-cells and activation of Tregs, illustrated in a schematic. *p<0.05. NS: non-significant. N=10. Scale bars are 30 μm.

### SEQUENCES

The nucleic and amino acid sequences are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file [Sequence_Listing, Dec. 28, 2020, 0.0187 in megabytes], which is incorporated by reference herein. In the accompanying sequence listing:

SEQ ID NO: 1 is an amino acid sequence of an exemplary human TIPE protein.

SEQ ID NO: 2 is an amino acid sequence of an exemplary mouse TIPE protein.

SEQ ID NO: 3 is an exemplary nucleic acid sequence encoding SEQ ID NO: 1.

SEQ ID NO: 4 is an exemplary nucleic acid sequence encoding SEQ ID NO: 2.

SEQ ID NO: 5 is a nucleic acid sequence of an exemplary pcDNA2-CD68 promoter and enhancer.

SEQ ID NO: 6 is a nucleic acid sequence of an exemplary CD11b promoter.

### DETAILED DESCRIPTION OF SEVERAL EMBODIMENTS

Macrophages that display the classic pro-inflammatory phenotype are said to have an “M1” polarization, whereas “M2” polarized macrophages are responsible for wound healing and tissue-remodeling functions. The degree to which a given macrophage bears M1 or M2 characteristics is termed “polarization” (Taylor et al., Annu Rev Immunol 23, 901-944 (2005)). It is now known that macrophages can actually “polarize” into a wide spectrum of phenotypes that do not fit rigidly into the definition of “M1” or “M2” (Nahrendorf and Swirski, Circ Res 119, 414-417 (2016)). In general, many microenvironmental signals, together with epigenetic changes, seem to influence macrophage activation and function Ginhoux, et al., Nat Immunol 17, 34-40 (2016)). Macrophages play a crucial role in T1D onset (8). The role of macrophages in T1D is believed to primarily result from their M1-like polarization, especially with their guidance of T-cells towards becoming anti-beta-cell cytotoxic T-cells (Jun, et al. J Exp Med 189, 347-358 (1999)). Very recently, an anti-autoimmune role for M2-like polarization of macrophages has been shown in T1D. Adoptive transfer of M2 macrophages prevented T1D in NOD mice, possibly through suppression of macrophage-mediated T-cell proliferation (Parsa et al., Diabetes 61, 2881-2892 (2012)). In another study, a novel mechanistic link between NADPH oxidase-derived ROS and macrophage phenotypes was revealed, demonstrating that superoxide is an important factor in macrophage polarization and a regulator of the onset of T1D (Padgett, et al., Diabetes 64, 937-946 (2015)).

Macrophage polarization is orchestrated by a complex network of signaling molecules, transcription factors, and post-transcriptional and epigenetic regulatory molecules. For example, activated canonical STAT signaling pathways direct macrophage differentiation toward the M1 phenotype via STAT1, or direct it toward the M2 phenotype via STAT6 (Sica, V. Bronte, J Clin Invest 117, 1155-1166 (2007)). TNF-alpha-induced protein 8-like 2 (TIPE2) (Sun et al., Cell 133, 415-426 (2008)), a negative regulator of inflammation, has been shown to be a trigger for M2 polarization of macrophages (Ding, et al., Cell Physiol Biochem 37, 2425-2433 (2015); Li, et al., Cell Physiol Biochem 38, 330-339 (2016); Li, Tumour Biol, (2015); Lou et al., PLoS One 9, e96508 (2014)). Nevertheless, a clinically translatable approach to suppress autoimmune T1D through the induction of pancreas-specific M2-like macrophage polarization has not been previously demonstrated.

It is disclosed herein that a vector comprising a macrophage specific promoter operably linked to a nucleic acid molecule encoding TNF-alpha-induced protein 8-like 2 (TIPE2) can be administered to a subject to induce M2-like macrophage polarization. In some embodiments, the vector is administered locally to a pancreas of the subject to induce pancreas-specific M2-like macrophage polarization. The disclosed vectors and methods are of use for the treatment of diabetes.

### Terms

Unless otherwise noted, technical terms are used according to conventional usage. Definitions of many common terms in molecular biology may be found in Krebs et al. (eds.), Lewin's genes XII, published by Jones & Bartlett Learning, 2017. The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Unless otherwise indicated, “about” means with five percent. Dates of GENBANK® Accession Nos. referred to herein are the sequences available at least as early as Dec. 31, 2019. All references, patent applications and publications, and GENBANK® Accession numbers cited herein are incorporated by reference.

In order to facilitate review of the various embodiments of this disclosure, the following explanations of specific terms are provided:

Alpha (α) cells: Mature glucagon producing endocrine cells. In vivo, these cells are found in the pancreatic islets of Langerhans.

Beta (β) cells: Mature insulin producing endocrine cells. In vivo, these cells are found in the pancreatic islets of Langerhans,

Delta (δ) cells: Mature somatostatin producing endocrine cells. In vivo, these cells are found in the pancreatic islets of Langerhans.

PP cells: Mature pancreatic polypeptide (PP) producing endocrine cells. In vivo, these cells are found in the pancreatic islets of Langerhans.

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 11 recognized serotypes of AAV (AAV1-11).

Administration: To provide or give a subject an agent by any effective route. Exemplary routes of administration include, but are not limited to, oral, injection (such as subcutaneous, intramuscular, intradermal, intraperitoneal, intravenous, and intraductal), sublingual, rectal, transdermal, intranasal, vaginal and inhalation routes. In some embodiments, administration is to a pancreatic duct.

Agent: Any polypeptide, compound, small molecule, organic compound, salt, polynucleotide, or other molecule of interest. Agent can include a therapeutic agent, a diagnostic agent or a pharmaceutical agent. A therapeutic agent is a substance that demonstrates some therapeutic effect by restoring or maintaining health, such as by alleviating the symptoms associated with a disease or physiological disorder, or delaying (including preventing) progression or onset of a disease,′ such as T1D. An agent can be a viral vector encoding a polypeptide of interest.

Amplification: Of a nucleic acid molecule (such as, a DNA or RNA molecule) refers to use of a technique that increases the number of copies of a nucleic acid molecule in a specimen. An example of amplification is the polymerase chain reaction, in which a biological sample collected from a subject is contacted with a pair of oligonucleotide primers, under conditions that allow for the hybridization of the primers to a nucleic acid template in the sample. The primers are extended under suitable conditions, dissociated from the template, and then re-annealed, extended, and dissociated to amplify the number of copies of the nucleic acid. The product of amplification may be characterized by electrophoresis, restriction endonuclease cleavage patterns, oligonucleotide hybridization or ligation, and/or nucleic acid sequencing using standard techniques. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in EP-A-320 308; gap filling ligase chain reaction amplification, as disclosed in U.S. Pat. No. 5,427,930; and NASBA™ RNA transcription-free amplification, as disclosed in U.S. Pat. No. 6,025,134.

Anti-diabetic lifestyle modifications: Changes to lifestyle, habits, and practices intended to alleviate the symptoms of diabetes or pre-diabetes. Obesity and sedentary lifestyle may both independently increase the risk of a subject developing type II diabetes, so anti-diabetic lifestyle modifications include those changes that will lead to a reduction in a subject's body mass index (BMI), increase physical activity, or both. Specific, non-limiting examples include the lifestyle interventions described in Diabetes Care, 22(4):623-34 at pages 626-27, herein incorporated by reference.

Conservative Substitutions: Modifications of a polypeptide that involve the substitution of one or more amino acids for amino acids having similar biochemical properties that do not result in change or loss of a biological or biochemical function of the polypeptide are designated “conservative” substitutions. These conservative substitutions are likely to have minimal impact on the activity of the resultant protein. Table 1 shows amino acids that can be substituted for an original amino acid in a protein, and which are regarded as conservative substitutions.

TABLE

| Original Residue | Conservative Substitutions |
|---|---|
| Ala | ser |
| Arg | lys |
| Asn | gln; his |
| Asp | glu |
| Cys | ser |
| Gln | asn |
| Glu | asp |
| Gly | pro |
| His | asn; gln |
| Ile | leu; val |
| Leu | ile; val |
| Lys | arg; gln; glu |
| Met | leu; ile |
| Phe | met; leu; tyr |
| Ser | thr |
| Thr | ser |
| Trp | tyr |
| Tyr | trp; phe |
| Val | ile; leu |

One or more conservative changes, or up to ten conservative changes (such as two substituted amino acids, three substituted amino acids, four substituted amino acids, or five substituted amino acids, etc.) can be made in the polypeptide without changing a biochemical function of the protein, such as TIPE2.

Diabetes mellitus: A group of metabolic diseases in which a subject has high blood sugar, either because the pancreas does not produce enough insulin, or because cells do not respond to the insulin that is produced. T1D results from the body's failure to produce insulin. This form has also been called “insulin-dependent diabetes mellitus” (IDDM) or “juvenile diabetes.” T1D is characterized by loss of the insulin-producing β cells, leading to insulin deficiency. This type can be further classified as immune-mediated or idiopathic. T2D results from insulin resistance, a condition in which cells fail to use insulin properly, sometimes combined with an absolute insulin deficiency. This form is also called “non insulin-dependent diabetes mellitus” (NIDDM) or “adult-onset diabetes.” The defective responsiveness of body tissues to insulin is believed to involve the insulin receptor. Diabetes mellitus is characterized by recurrent or persistent hyperglycemia, and is diagnosed by demonstrating any one of:
a. Fasting plasma glucose level ≥7.0 mmol/l (126 mg/dl);
b. Plasma glucose ≥11.1 mmol/l (200 mg/dL) two hours after a 75 g oral glucose load as in a glucose tolerance test;
c. Symptoms of hyperglycemia and casual plasma glucose ≥11.1 mmol/l (200 mg/dl);
d. Glycated hemoglobin (Hb A1C)≥6.5%

Endocrine: Tissue which secretes regulatory hormones directly into the bloodstream without the need for an associated duct system.

Enhancer: A nucleic acid sequence that increases the rate of transcription by increasing the activity of a promoter.

Expand: A process by which the number or amount of cells is increased due to cell division. Similarly, the terms “expansion” or “expanded” refers to this process. The terms “proliferate,” “proliferation” or “proliferated” may be used interchangeably with the words “expand,” “expansion,” or “expanded.”

Expressed: Translation of a nucleic acid into a protein. Proteins may be expressed and remain intracellular, become a component of the cell surface membrane, or be secreted into the extracellular matrix or medium.

Exocrine: Secretory tissue which distributes its products, such as enzymes, via an associated duct network. The exocrine pancreas is the part of the pancreas that secretes enzymes required for digestion. The exocrine cells of the pancreas include the centroacinar cells and basophilic cells, which produce secretin and cholecystokinin.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Heterologous: A heterologous sequence is a sequence that is not normally (in the wild-type sequence) found adjacent to a second sequence. In one embodiment, the sequence is from a different genetic source, such as a virus or organism, than the second sequence.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Insulin: A protein hormone involved in the regulation of blood sugar levels that is produced by pancreatic beta cells. In vivo, insulin is produced as a precursor proinsulin, consisting of the B and A chains of insulin linked together via a connecting C-peptide. Insulin itself includes only the B and A chains. Exemplary insulin sequences are provided in GENBANK® Accession NO. NM_000207.2 (human) and NM_008386.3 (mouse), as available on Apr. 1, 2015, and are incorporated by reference herein. Exemplary nucleic acid sequences encoding insulin are provided in GENBANK® Accession No: NM_000207.2 (human) and NM_008386.3 (mouse), as available on Apr. 1, 2015, and are incorporated by reference herein. The term insulin also encompasses species variants, homologues, allelic forms, mutant forms, and equivalents thereof, including conservative substitutions, additions, deletions therein not adversely affecting the structure of function.

Islets of Langerhans: Small discrete clusters of pancreatic endocrine tissue. In vivo, in an adult mammal, the islets of Langerhans are found in the pancreas as discrete clusters (islands) of pancreatic endocrine tissue surrounded by the pancreatic exocrine (or acinar) tissue. In vivo, the islets of Langerhans consist of the a cells, β cells, δ cells, PP cells, and c cells. Histologically, in rodents, the islets of Langerhans consist of a central core of β cells surrounded by an outer layer of a cells, δ cells, and PP cells. The structure of human islets of Langerhans is different and distinct from rodents. The islets of Langerhans are sometimes referred to herein as “islets.”

Isolated: An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids. An isolated cell type has been substantially separated from other cell types, such as a different cell type that occurs in an organ. A purified cell or component can be at least 90%, at least 95%, at least 96%, at least 97%, at least 98%, or at least 99% pure.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes.

Macrophage: A type of white blood cell that phagocytoses and degrades cellular debris, foreign substances, microbes, and cancer cells. In addition to their role in phagocytosis, these cells play an important role in development, tissue maintenance and repair, and in both innate and adaptive immunity in that they recruit and influence other cells including immune cells such as lymphocytes. Macrophages can exist in many phenotypes, including phenotypes that have been referred to as M1 and M2. Macrophages that perform primarily pro-inflammatory functions are called M1 macrophages (CD86+/CD68+), whereas macrophages that decrease inflammation and encourage and regulate tissue repair are called M2 macrophages (CD206+/CD68+). The markers that identify the various phenotypes of macrophages vary among species. The degree to which a given macrophage bears M1 or M2 characteristics is termed “polarization” (Taylor et al., Annu Rev Immunol 23, 901-944 (2005)). It is now known that macrophages can actually “polarize” into a wide spectrum of phenotypes that do not fit rigidly into the definition of “M1” or “M2” (Nahrendorf and Swirski, Circ Res 119, 414-417 (2016)). “M2 polarization” indicates that the macrophages have M2 characteristics, such as, but not limited to, expression of CD68 and/or CD206, and having anti-inflammatory activity. Macrophage polarization is a process by which macrophages adopt different functional programs in response to the signals from their microenvironment. Markers are used to determine the polarization status and alteration of function.

Musculoaponeurotic fibrosarcoma oncogene homolog A (MafA): MAFA is a transcription factor that binds RIPE3b, a conserved enhancer element that regulates pancreatic beta cell-specific expression of the insulin gene (INS; MIM 176730) (Olbrot et al., 2002). MafA is referred in the art as aliases; v-maf musculoaponeurotic fibrosarcoma oncogene homolog A (avian), hMafA; RIPE3b1; MAFA. Exemplary MafA proteins are the MafA protein of GENBANK® Accession No: NM_194350 (mouse) (SEQ ID NO:3 32 of U.S. Published Patent Application No. 2011/0280842) or NP_963883.2 (Human)(SEQ ID NOs: 33 and 32 of U.S. Published Patent Application No. 2011/0280842); GeneID No: 389692, which are all incorporated by reference. The term MafA also encompasses species variants, homologues, allelic forms, mutant forms, and equivalents thereof, including conservative substitutions, additions, deletions that do not adversely affecting the structure of function. The term “MafA”, or “MafA” protein” as used herein refers to a polypeptide having a naturally occurring amino acid sequence of a MafA” protein or a fragment, variant, or derivative thereof retains the ability of the naturally occurring protein to bind to DNA and activate gene transcription of Glut2 and pyruvate carboxylase, and other genes such as Glut2, Pdx-1, Nkx6.1, GLP-1 receptor, prohormone convertase-1/3 as disclosed in Wang et al., Diabetologia. 2007 February; 50(2): 348-358, which is incorporated herein by reference. Exemplary MafA nucleic acids are GENBANK® Accession No: NM_201589 (human) (SEQ ID NO:36 of U.S. Published Patent Application No. 2011/0280842) and GENBANK® Accession No: NM_194350 (mouse) (SEQ ID NO: 39 of U.S. Published Patent Application No. 2011/0280842), which are all incorporated by reference. In addition to naturally-occurring allelic variants of the MafA sequences that may exist in the population, it will be appreciated that, as is the case for virtually all proteins, a variety of changes can be introduced into the sequences of SEQ ID NO: 3 of U.S. Published Patent Application No. 2011/0280842 or SEQ ID NO: 33 of U.S. Published Patent Application No. 2011/0280842 (referred to as “wild type” sequences) without substantially altering the functional (biological) activity of the polypeptides. Such variants are included within the scope of the terms “MafA”, “MafA protein”, etc. U.S. Published Patent Application No. 2011/0280842 and all of its referenced GENBANK entries are incorporated herein by reference.

Neurogenin (Ngn)3: Neurogenin-3 (also known as NEUROG3) is expressed in endocrine progenitor cells and is required for endocrine cell development in the pancreas and intestine. It belongs to a family of basic helix-loop-helix transcription factors involved in the determination of neural precursor cells in the neuroectoderm. Ngn3 is referred in the art as aliases; Neurogenin 3; Atoh5; Math4B; bHLHa7; NEUROG3. Exemplary Ngn3 proteins are provided in GENBANK® Accession No: NM_009719 (mouse) and SEQ ID NO:2 of U.S. Published Patent Application No. 2011/0280842, both incorporated by reference herein or GENBANK® Accession No: NP_033849.3 (Human) and SEQ ID NO: 32 of U.S. Published Patent Application No. 2011/0280842, both incorporated by reference herein; GeneID No: 50674. The term Ngn3 also encompasses species variants, homologues, allelic forms, mutant forms, and equivalents thereof, including conservative substitutions, additions, deletions therein not adversely affecting the structure of function. Human Ngn3 is encoded by nucleic acid corresponding to GENBANK® Accession No: NM_020999 (human), SEQ ID NO:35 of U.S. Published Patent Application No. 2011/0280842 or NM_009719 (mouse), SEQ ID NO: 38 of U.S. Published Patent Application No. 2011/0280842. U.S. Published Patent Application No. 2011/0280842 and these GENBANK® Accession Nos. are incorporated by reference herein. The term “Ngn3”, or “Ngn3 protein” as used herein refers to a polypeptide having a naturally occurring amino acid sequence of a Ngn3 protein or a fragment, variant, or derivative thereof that retains the ability of the naturally occurring protein to bind to DNA and activate gene transcription of NeuroD, Delta-like 1(D111), HeyL, insulinoma-assiciated-1 (IA1), Nk2.2, Notch, HesS, Isl1, Somatostatin receptor 2 (Sstr2) and other genes as disclosed in Serafimidis et al., Stem cells; 2008; 26; 3-16, which is incorporated herein in its entirety by reference. In addition to naturally-occurring allelic variants of the Ngn3 sequences that may exist in the population, it will be appreciated that, as is the case for virtually all proteins, a variety of changes can be introduced into a wild-type sequence (listed above in GENBANK® entries) without substantially altering the functional (biological) activity of the polypeptides. Such variants are included within the scope of the terms “Ngn3,” “Ngn3 protein,” etc.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, such as a “recombinant polypeptide.” A recombinant nucleic acid may serve a non-coding function (such as a promoter, origin of replication, ribosome-binding site, etc.) as well.

A first sequence is an “antisense” with respect to a second sequence if a polynucleotide whose sequence is the first sequence specifically hybridizes with a polynucleotide whose sequence is the second sequence.

Terms used to describe sequence relationships between two or more nucleotide sequences or amino acid sequences include “reference sequence,” “selected from,” “comparison window,” “identical,” “percentage of sequence identity,” “substantially identical,” “complementary,” and “substantially complementary.”

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, for example, by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see for example, Current Protocols in Molecular Biology (Ausubel et al., eds 1995 supplement)).

One example of a useful algorithm is PILEUP. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, such as version 7.0 (Devereaux et al., Nuc. Acids Res. 12:387-395, 1984.

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (http://www.ncbi.nlm.nih.gov/). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989).

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

ORF (open reading frame): A series of nucleotide triplets (codons) coding for amino acids without any termination codons. These sequences are usually translatable into a peptide.

Pancreatic endocrine cell: An endocrine cell of pancreatic origin that produces one or more pancreatic hormone, such as insulin, glucagon, somatostatin, or pancreatic polypeptide. Subsets of pancreatic endocrine cells include the a (glucagon producing), 13 (insulin producing) 6 (somatostatin producing) or PP (pancreatic polypeptide producing) cells. In some embodiments, pancreatic endocrine cells produce ghrelin. Additional subsets produce more than one pancreatic hormone, such as, but not limited to, a cell that produces both insulin and glucagon, or a cell that produces insulin, glucagon, and somatostatin, or a cell that produces insulin and somatostatin.

Pancreas duodenal homeobox protein (Pdx)1: Pdx1 protein is a transcriptional activator of several genes, including insulin, somatostatin, glucokinase, islet amyloid polypeptide, and glucose transporter type 2 (GLUT2). Pdx1 is a nuclear protein is involved in the early development of the pancreas and plays a major role in glucose-dependent regulation of insulin gene expression. Defects in the gene encoding the Pdx1 preotein are a cause of pancreatic agenesis, which can lead to early-onset insulin-dependent diabetes mellitus (NIDDM), as well as maturity onset diabetes of the young type 4 (MODY4). Pdx1 is referred in the art as aliases; pancreatic and duodenal homeobox 1, IDX-1, STF-1, PDX-1, MODY4, Ipf1. Exemplary Pdx1 proteins are shown in GENBANK® Accession No. NM_008814 (mouse) (SEQ ID NO:1 of U.S. Published Patent Application No. 2011/0280842) or GENBANK® Accession No. NP_000200.1 (Human)(SEQ ID NO: 31 of U.S. Published Patent Application No. 2011/0280842), or Gene ID: 3651, which are all incorporated herein by reference. The term Pdx1 also encompasses species variants, homologues, allelic forms, mutant forms, and equivalents thereof, including conservative substitutions, additions, deletions therein not adversely affecting the structure of function. Exemplary nucleic acid sequences are shown in GENBANK® Accession No NM_000209 (human) (SEQ ID NO:34 of U.S. Published Patent Application No. 2011/0280842) or GENBANK® Accession No NM_008814 (mouse) (SEQ ID NO: 37 of U.S. Published Patent Application No. 2011/0280842), which are all incorporated by reference. The term “Pdx1”, or “Pdx1 protein” as used herein refers to a polypeptide having a naturally occurring amino acid sequence of a Pdx1 protein or a fragment, variant, or derivative thereof that at least in part retains the ability of the naturally occurring protein to bind to DNA and activate gene transcription of insulin, somatostatin, glucokinase, islet amyloid polypeptide, and glucose transporter type 2 (GLUT2). In addition to naturally-occurring allelic variants of the Pdx1 sequences that may exist in the population, it will be appreciated that, as is the case for virtually all proteins, a variety of changes can be introduced into a wild type sequence (see the listed GENBANK® entries) without substantially altering the functional (biological) activity of the polypeptides. Such variants are included within the scope of the terms “Pdx1”, “Pdx1 protein,” etc. The listed GENBANK® Accession Nos. and of U.S. Published Patent Application No. 2011/0280842 are incorporated by reference herein.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical agent: A chemical compound or a composition capable of inducing a desired therapeutic or prophylactic effect when properly administered to a subject or a cell. “Incubating” includes a sufficient amount of time for a drug to interact with a cell. “Contacting” includes incubating a drug in solid or in liquid form with a cell.

Pre-diabetes: A state in which some, but not all, of the criteria for diabetes are met. For example, a subject can have impaired fasting glycaemia or impaired fasting glucose (IFG). Subjects with fasting glucose levels of 100 or higher but less than 126 mg/dl (6.1 to 6.9 mmol/1) are considered to have impaired fasting glucose. Subjects with plasma glucose at or above 140 mg/dL (7.8 mmol/L), but not over 200 mg/dL (11.1 mmol/L), two hours after a 75 g oral glucose load are considered to have impaired glucose tolerance. Subjects with an elevated HbA1c level (5.7%-6.5%) are considered pre-diabetic. Pre-diabetes can be diagnosed by:

A1C 5.7% to <6.5%

Impaired fasting glucose: fasting glucose ≥100 but <126 mg/dL.

Impaired glucose tolerance: 2-h plasma glucose ≥140 but <200 mg/dL during an OGTT, when the test is performed as described by the World Health Organization, using a glucose load containing the equivalent of 1.75 mg/kg (max 75 g) anhydrous glucose dissolved in water.

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those that are recombinantly or synthetically produced.

The term “polypeptide fragment” refers to a portion of a polypeptide which exhibits at least one useful epitope. The term “functional fragments of a polypeptide” refers to all fragments of a polypeptide that retain an activity of the polypeptide. Biologically functional fragments, for example, can vary in size from a polypeptide fragment as small as an epitope capable of binding an antibody molecule to a large polypeptide capable of participating in the characteristic induction or programming of phenotypic changes within a cell. An “epitope” is a region of a polypeptide capable of binding an immunoglobulin generated in response to contact with an antigen. Thus, smaller peptides containing the biological activity of insulin, or conservative variants of the insulin, are thus included as being of use.

The term “soluble” refers to a form of a polypeptide that is not inserted into a cell membrane.

The term “substantially purified polypeptide” as used herein refers to a polypeptide which is substantially free of other proteins, lipids, carbohydrates or other materials with which it is naturally associated. In one embodiment, the polypeptide is at least 50%, for example at least 80% free of other proteins, lipids, carbohydrates or other materials with which it is naturally associated. In another embodiment, the polypeptide is at least 90% free of other proteins, lipids, carbohydrates or other materials with which it is naturally associated. In yet another embodiment, the polypeptide is at least 95% free of other proteins, lipids, carbohydrates or other materials with which it is naturally associated.

Conservative substitutions replace one amino acid with another amino acid that is similar in size, hydrophobicity, etc. Variations in the cDNA sequence that result in amino acid changes, whether conservative or not, should be minimized in order to preserve the functional and immunologic identity of the encoded protein. The immunologic identity of the protein may be assessed by determining if it is recognized by an antibody; a variant that is recognized by such an antibody is immunologically conserved. Any cDNA sequence variant can introduce no more than twenty, such as fewer than ten amino acid substitutions into the encoded polypeptide. Variant amino acid sequences may, for example, be 80, 90 or even 95% or 98% identical to the native amino acid sequence.

Preventing, treating or ameliorating a disease: “Preventing” a disease (such as T1D) refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Promoter: A promoter is an array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). A promoter that is “macrophage specific” is increased expression in macrophage cells as compared to other cell types, such as, but not limited to, lymphocytes, natural killer cells and neutrophils.

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, such as by genetic engineering techniques. Similarly, a recombinant protein is one encoded for by a recombinant nucleic acid molecule. In addition, a recombinant virus is a virus comprising sequence (such as genomic sequence) that is non-naturally occurring or made by artificial combination of at least two sequences of different origin. The term “recombinant” also includes nucleic acids, proteins and viruses that have been altered solely by addition, substitution, or deletion of a portion of a natural nucleic acid molecule, protein or virus. As used herein, “recombinant AAV” refers to an AAV particle in which a recombinant nucleic acid molecule (such as a recombinant nucleic acid molecule encoding Pdx1 and MafA) has been packaged.

Sequence identity of amino acid sequences: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of proteins, such as TIPE2, MafA or Pdx1 are typically characterized by possession of at least about 75%, for example at least about 80%, 90%, 95%, 96%, 97%, 98% or 99% sequence identity counted over the full length alignment with the amino acid sequence of the antibody using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Subject: Any mammal, such as humans, non-human primates, pigs, sheep, cows, rodents and the like which is to be the recipient of the particular treatment. In two non-limiting examples, a subject is a human subject or a murine subject. In some embodiments, the subject has T1D.

Therapeutic agent: Used in a generic sense, it includes treating agents, prophylactic agents, and replacement agents. A therapeutic agent can be a nucleic acid molecule encoding TIPE2. A therapeutic agent also can be a nucleic acid molecule encoding MafA and Pdx-1, or a vector encoding these factors.

Therapeutically effective amount: A quantity of a specified pharmaceutical or therapeutic agent (e.g. a recombinant AAV) sufficient to achieve a desired effect in a subject, or in a cell, being treated with the agent, such as increasing M2 macrophages or increasing insulin production in a subject with diabetes. The effective amount of the agent will be dependent on several factors, including, but not limited to the subject or cells being treated, and the manner of administration of the therapeutic composition.

Transduced and Transformed: A virus or vector “transduces” a cell when it transfers nucleic acid into the cell. A cell is “transformed” or “transfected” by a nucleic acid transduced into the cell when the DNA becomes stably replicated by the cell, either by incorporation of the nucleic acid into the cellular genome, or by episomal replication.

Numerous methods of transfection are known to those skilled in the art, such as: chemical methods (e.g., calcium-phosphate transfection), physical methods (e.g., electroporation, microinjection, particle bombardment), fusion (e.g., liposomes), receptor-mediated endocytosis (e.g., DNA-protein complexes, viral envelope/capsid-DNA complexes) and by biological infection by viruses such as recombinant viruses {Wolff, J. A., ed, Gene Therapeutics, Birkhauser, Boston, USA (1994)}. In the case of infection by retroviruses, the infecting retrovirus particles are absorbed by the target cells, resulting in reverse transcription of the retroviral RNA genome and integration of the resulting provirus into the cellular DNA. Methods for the introduction of genes into the pancreatic endocrine cells are known (e.g. see U.S. Pat. No. 6,110,743, herein incorporated by reference). These methods can be used to transduce a pancreatic endocrine cell produced by the methods described herein, or an artificial islet produced by the methods described herein.

Genetic modification of the target cell is an indicium of successful transfection. “Genetically modified cells” refers to cells whose genotypes have been altered as a result of cellular uptakes of exogenous nucleotide sequence by transfection. A reference to a transfected cell or a genetically modified cell includes both the particular cell into which a vector or polynucleotide is introduced and progeny of that cell.

Transgene: An exogenous gene supplied by a vector.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like. In some embodiments herein, the vector is an adenovirus vector or an AAV vector.

Virus: Microscopic infectious organism that reproduces inside living cells. A virus consists essentially of a core of a single nucleic acid surrounded by a protein coat and has the ability to replicate only inside a living cell. “Viral replication” is the production of additional virus by the occurrence of at least one viral life cycle. Viral vectors are known in the art, and include, for example, adenovirus, AAV, lentivirus and herpes virus.

It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

### Vectors

Disclosed herein are vectors, such as a viral vector, such as a retroviral vector, an adenoviral vector, or an adeno-associated vector (AAV) that encodes TIPE2. These vectors include a nucleotide acid molecule operably linked to a macrophage specific promoter. Viral vectors include an attenuated or defective DNA or RNA viruses, including, but not limited to, adenovirus or adeno-associated virus (AAV). Defective viruses, that entirely or almost entirely lack viral genes, can be used. Use of defective viral vectors allows for administration to specific cells without concern that the vector can infect other cells. In some examples, the vector is an attenuated adenovirus vector, such as the vector described by Stratford-Perricaudet et al. (J. Clin. Invest., 90:626-630 1992; La Salle et al., Science 259:988-990, 1993); and a defective adeno-associated virus vector (Samulski et al., J. Virol., 61:3096-3101, 1987; Samulski et al., J. Virol., 63:3822-3828, 1989; Lebkowski et al., Mol. Cell. Biol., 8:3988-3996, 1988).

Suitable vectors are known in the art, and include viral vectors such as retroviral, lentiviral, adenoviral vectors, and AAV. In specific, non-limiting examples, the vector is a lentiviral vector, gammaretroviral vector, self-inactivating retroviral vector, adenoviral vector, or adeno-associated vector (AAV).

Adenoviral vectors and/or adeno-associated viral vectors can be used in the methods disclosed herein. AAV belongs to the family Parvoviridae and the genus Dependovirus. AAV is a small, non-enveloped virus that packages a linear, single-stranded DNA genome. Both sense and antisense strands of AAV DNA are packaged into AAV capsids with equal frequency. In some embodiments the AAV DNA includes a nucleic acid encoding TIPE2, operably linked to a macrophage specific promoter. Further provided are recombinant vectors, such as recombinant adenovirus vectors and recombinant adeno-associated virus (rAAV) vectors comprising a nucleic acid molecule disclosed herein. In some embodiments, the AAV is rAAV8 and/or AAV2. However, the AAV serotype can be any other suitable AAV serotype, such as AAV1, AAV2, AAV3, AAV4, AAV5, AAV6, AAV7, AAV9, AAV10, AAV11 or AAV12, or a hybrid of two or more AAV serotypes (such as, but not limited to AAV2/1, AAV2/7, AAV2/8 or AAV2/9).

The AAV genome is characterized by two inverted terminal repeats (ITRs) that flank two open reading frames (ORFs). In the AAV2 genome, for example, the first 125 nucleotides of the ITR are a palindrome, which folds upon itself to maximize base pairing and forms a T-shaped hairpin structure. The other 20 bases of the ITR, called the D sequence, remain unpaired. The ITRs are cis-acting sequences important for AAV DNA replication; the ITR is the origin of replication and serves as a primer for second-strand synthesis by DNA polymerase. The double-stranded DNA formed during this synthesis, which is called replicating-form monomer, is used for a second round of self-priming replication and forms a replicating-form dimer. These double-stranded intermediates are processed via a strand displacement mechanism, resulting in single-stranded DNA used for packaging and double-stranded DNA used for transcription. Located within the ITR are the Rep binding elements and a terminal resolution site (TRS). These features are used by the viral regulatory protein Rep during AAV replication to process the double-stranded intermediates. In addition to their role in AAV replication, the ITR is also essential for AAV genome packaging, transcription, negative regulation under non-permissive conditions, and site-specific integration (Daya and Berns, Clin Microbiol Rev 21(4):583-593, 2008). In some embodiments, these elements are included in the AAV vector.

The left ORF of AAV contains the Rep gene, which encodes four proteins—Rep78, Rep 68, Rep52 and Rep40. The right ORF contains the Cap gene, which produces three viral capsid proteins (VP1, VP2 and VP3). The AAV capsid contains 60 viral capsid proteins arranged into an icosahedral symmetry. VP1, VP2 and VP3 are present in a 1:1:10 molar ratio (Daya and Berns, Clin Microbiol Rev 21(4):583-593, 2008). In some embodiments, these elements are included in the AAV vector

AAV vectors can be used for gene therapy. Exemplary AAV of use are AAV2, AAV5, AAV6, AAV8 and AAV9. Adenovirus, AAV2 and AAV8 are capable of transducing cells in the pancreas. Thus, any of a rAAV2 or rAAV8 vector can be used in the methods disclosed herein. In addition, rAAV6 and rAAV9 vectors are also of use, as these are capable of transducing macrophages. In one non-limiting example, the vector is an rAAV6 vector.

In some embodiments, the AAV vector includes only the ITR and the gene of interest, specifically the macrophage promoter operably linked to the nucleic acid molecule encoding TIPE2. In these embodiments, Rep and Cap are provided by a host cell, or on another vector, for in vitro production of the virus, but the resulting virus does not include nucleic acid molecules encoding Rep or Cap. In some embodiments, rAAV particles are generated by transfecting producer cells with a plasmid (AAV cis-plasmid) containing a cloned recombinant AAV genome composed of foreign DNA flanked by the 145 nucleotide-long AAV ITRs, and a separate construct expressing in trans the viral rep and cap genes. The adenovirus helper factors, such as E1A, E1B, E2A, E4ORF6 and VA RNAs, can be provided by either adenovirus infection or transfecting into production cells a third plasmid that provides these adenovirus helper factors. In some embodiments, HEK293 cells are utilized. These are commonly used AAV production cells, which include the E1A/E1b gene; the helper factors that need to be provided are E2A, E4ORF6 and VA RNAs. Methods, vectors, and cells of use, are disclosed, for example, in U.S. Pat. Nos. 6,566,118; 6,686,200; 6,924,128, 7,091,029, and 7,208,315, which are all incorporated herein by reference.

In some embodiments, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

The minigene, rep sequences, cap sequences, and helper functions required for producing a rAAV can be delivered to the packaging host cell in the form of any genetic element which transfer the sequences carried thereon. The selected genetic element may be delivered by any suitable method, including those described herein. The methods used to construct vectors are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, J. Virol., 70:520-532 (1993), U.S. Pat. No. 5,478,745, and PCT Publication No. and WO 2005/033321, incorporated herein by reference. In some embodiments, selected AAV components can be readily isolated using techniques available to those of skill in the art from an AAV serotype, including AAV6. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GENBANK®.

Although AAV infects humans and some other primate species, it is not known to cause disease and elicits a very mild immune response. Gene therapy vectors that utilize AAV can infect both dividing and quiescent cells and persist in an extrachromosomal state without integrating into the genome of the host cell. AAV6 preferentially infects macrophages. Because of the advantageous features of AAV, an rAAV are of use in the methods disclosed herein. However, this is not limiting.

AAV possesses several additional desirable features for a gene therapy vector, including the ability to bind and enter target cells, enter the nucleus, the ability to be expressed in the nucleus for a prolonged period of time, and low toxicity. AAV can be used to transfect cells, and suitable vector are known in the art, see for example, U.S. Published Patent Application No. 2014/0037585, incorporated herein by reference. Methods for producing rAAV suitable for gene therapy are well known in the art (see, for example, U.S. Published Patent Application Nos. 2012/0100606; 2012/0135515; 2011/0229971; and 2013/0072548; and Ghosh et al., Gene Ther 13(4):321-329, 2006), and can be utilized with the methods disclosed herein.

In some embodiments, the vector is an rAAV6 vector, an rAAV8 vector, an rAAV2 vector, or an rAAV9 vector. rAAV6 vectors are disclosed, for example, in U.S. Pat. No. 9,439,979. rAAV6 vectors are also disclosed in Xie et al, Structure-function analysis of receptor-binding in adeno-associated virus serotype 6 (AAV-6), Virology 420 (2011) 10-19, incorporated by reference herein. rAAV vectors are disclosed, for example, in U.S. Pat. No. 6,156,303, which is incorporated by reference herein. An exemplary AAV6 nucleic acid sequence is shown, for example, in SEQ ID NO: 2. The location and sequence of the capsid, rep 68/78, rep 40/52, VP1, VP2 and VP3 are disclosed in this U.S. Pat. No. 6,156,303. Hybrid vectors are also disclosed.

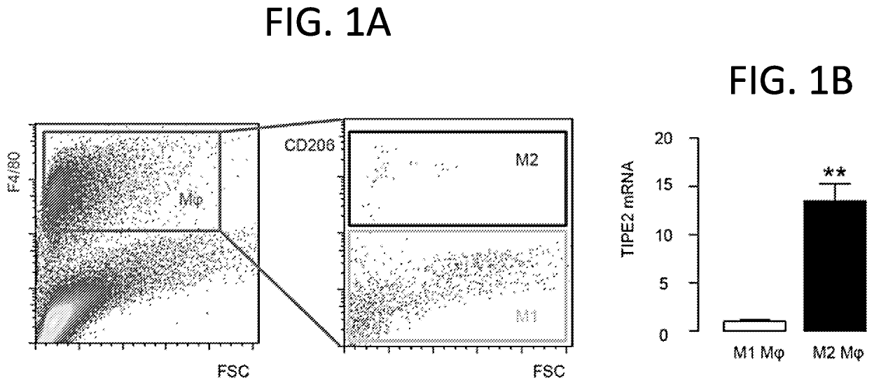

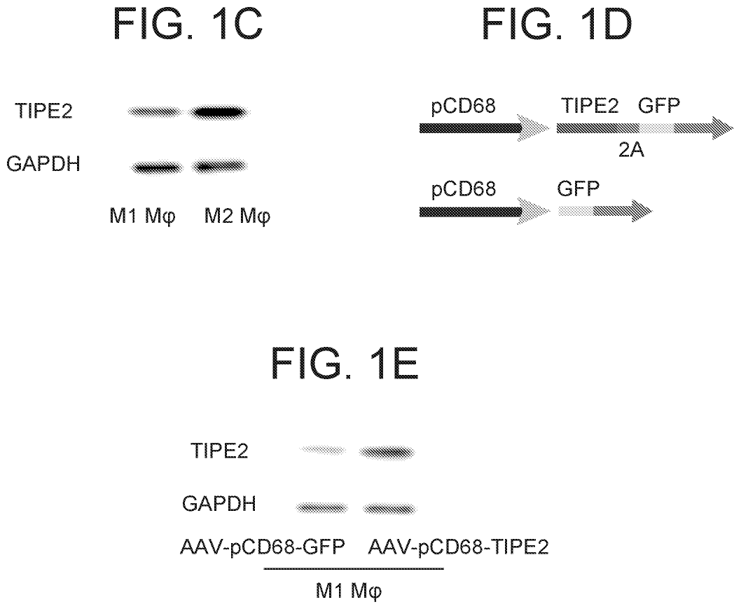

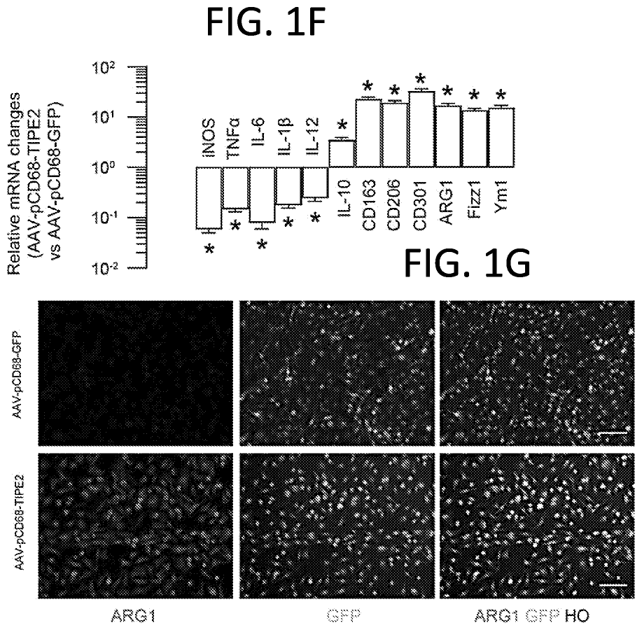

The vectors of use in the methods disclosed herein can contain nucleic acid sequences encoding an intact AAV capsid which may be from a single AAV serotype (e.g., AAV2, AAV, 6, AAV8 or AAV9). As disclosed in U.S. Pat. No. 6,156,303, vectors of use also can be recombinant, and thus can contain sequences encoding artificial capsids which contain one or more fragments of the AAV6 capsid fused to heterologous AAV or non-AAV capsid proteins (or fragments thereof). These artificial capsid proteins are selected from non-contiguous portions of the AAV2, AAV8 or AAV9 capsid or from capsids of other AAV serotypes. For example, a rAAV vector may have a capsid protein comprising one or more of the AAV6 capsid regions selected from the VP2 and/or VP3, or from VP1, or fragments thereof, see FIG. 1 of U.S. Pat. No. 6,156,303. In another example, it may be desirable to alter the start codon of the VP3 protein to GTG.

In some embodiments, a rAAV is generated having an AAV serotype 6 capsid. To produce the vector, a host cell which can be cultured that contains a nucleic acid sequence encoding an adeno-associated virus (AAV) serotype 6 capsid protein, or fragment thereof, as defined herein; a functional rep gene; a minigene composed of, at a minimum, AAV inverted terminal repeats (ITRs) and a transgene, such as a macrophage specific promoter, such as a CD68 or a CD11b promoter operably linked to a nucleic acid molecule encoding TIPE2; and sufficient helper functions to permit packaging in the AAV6 capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art. In some embodiments, a stable host cell will contain the required component(s) under the control of an inducible promoter. However, the required component(s) can be under the control of a constitutive promoter. Examples of suitable inducible and constitutive promoters are provided below. Similar methods can be used to generate a rAAV2, rAAV8 or rAAV9 vector and/or virion.

In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

The minigene, rep sequences, cap sequences, and helper functions required for producing a rAAV can be delivered to the packaging host cell in the form of any genetic element which transfer the sequences carried thereon. The selected genetic element may be delivered by any suitable method, including those described herein. The methods used to construct vectors are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, J. Virol., 70:520-532 (1993) and U.S. Pat. No. 5,478,745. In some embodiments, selected AAV components can be readily isolated using techniques available to those of skill in the art from an AAV serotype, including AAV6. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GENBANK®.

In some embodiments, the vector is a double-stranded self-complementary virus, or “scAAV vector.” scAAV vectors are disclosed in McCarty et al., 2001, Gene Ther. 8: 1248-1254; Carter PCT Publication No. WO 2001/011034; and Samulski, PCT Publication No. WO 2001/092551, all of which are incorporated by reference herein. As disclosed in PCT Publication No. “duplexed” DNA parvovirus vectors can be advantageously employed for gene delivery. Duplexed parvovirus can provide improved transduction to particle ratios, more rapid transgene expression, a higher level of transgene expression, and/or more persistent transgene expression. The duplexed parvovirus vectors can be used for gene delivery to host cells that are typically refractory to AAV transduction. Thus, duplexed parvovirus vectors, such as AAV6, can have a different host range than ssAAV (single-stranded) vectors.

These vectors are dimeric self-complementary (sc) polynucleotides (typically, DNA) packaged within a viral capsid, such as a parvovirus capsid, for example an AAV capsid, such as, but not limited to, AAV6. In some respects, the viral genome that is packaged within the capsid is essentially a “trapped” replication intermediate that cannot be resolved to produce the plus and minus polarity parvovirus DNA strands. Accordingly, the duplexed parvovirus vectors can circumvent the need for host cell mediated synthesis of complementary DNA inherent in conventional recombinant AAV (ssAAV) vectors.

This result is accomplished by allowing the virus to package essentially dimeric inverted repeats of the single-stranded parvovirus (e.g., ssAAV, such as ssAAV6) vector genome such that both strands, joined at one end, are contained within a single infectious capsid. Upon release from the capsid, the complementary sequences re-anneal to form transcriptionally active double-stranded DNA within the target cell.

The duplexed parvovirus vectors are fundamentally different from ssAAV vectors, and from the parent parvovirus in that the vDNA may form a double-stranded hairpin structure due to intrastrand base pairing, and that both DNA strands are encapsidated. Thus, the duplexed parvovirus vector is functionally similar to double-stranded DNA virus vectors rather than the parvovirus (e.g., ssAAV) from which it was derived.

The viral capsid may be from any parvovirus, either an autonomous parvovirus or dependovirus. In some embodiments, the viral capsid is an AAV capsid (e.g., an AAV2, AAV6, AAV or AAV9 capsid). The choice of parvovirus capsid may be based on a number of considerations as known in the art, e.g., the target cell type, the desired level of expression, the nature of the heterologous nucleotide sequence to be expressed, issues related to viral production, and the like. In a specific example, the capsid is an AAV6 capsid.

The parvovirus particle may be a “hybrid” particle in which the viral terminal repeats (TRs) and viral capsid are from different parvoviruses. In some embodiments, the viral TRs and capsid are from different serotypes of AAV (e.g., as described in PCT Publication No. WO 00/28004 and Chao et al., Molecular Therapy 2:619, 2000; the disclosures of which are incorporated herein in entirety). In some embodiments, the virus has a “chimeric” capsid (e.g., containing sequences from different parvoviruses) or a “targeted” capsid (e.g., a directed tropism) as described in these publications. As used herein, a “duplexed parvovirus particle” encompasses hybrid, chimeric and targeted virus particles. In some embodiments, the duplexed parvovirus particle has an AAV capsid, which may further be a chimeric or targeted capsid.

A duplexed parvovirus vector can be produced by any suitable method. In some embodiments, the template for producing the vDNA is one that gives rise to a duplexed, rather than monomeric vDNA (i.e., the majority of vDNA produced are duplexed) which has the capacity to form a double-stranded vDNA. In some embodiments, at least about 50%, 60%, 70%, 80%, 90%, 95%, 98%, 99% or more of the replication products from the template are duplexed. In one embodiment, the template is a DNA molecule comprising one or more terminal repeat (TR) sequences. The template also comprises a modified TR that cannot be resolved (i.e., nicked) by the parvovirus Rep proteins. During replication, the inability of Rep protein to resolve the modified TR will result in a stable intermediate with the two “monomers” covalently attached by the non-resolvable TR. This “duplexed” molecule may be packaged within the parvovirus (AAV) capsid to produce a novel duplexed parvovirus vector, such as a scAAV6 vector.

While not wishing to be held to any particular theory, it is likely that the virion genome is retained in a single-stranded form while packaged within the viral capsid. Upon release from the capsid during viral infection, the dimeric molecule “snaps back” or anneals to form a double-stranded molecule by intra-strand base pairing, with the non-resolvable TR sequence forming a covalently-closed hairpin structure at one end. This double-stranded vDNA obviates host cell mediated second-strand synthesis, which may be a rate-limiting step for AAV transduction.

In some embodiments, the template further comprises a heterologous nucleotide sequence(s) to be packaged for delivery to a target cell. According to this particular embodiment, the heterologous nucleotide sequence is located between the viral TRs at either end of the substrate. In further preferred embodiments, the parvovirus (e.g., AAV) cap genes and rep genes are deleted from the template (and the vDNA produced therefrom). This configuration maximizes the size of the heterologous nucleic acid sequence(s) that can be carried by the parvovirus capsid. This can be the macrophage specific promoter operably linked to a nucleic acid molecule encoding TIPE2.

In one embodiment, the template for producing the duplexed parvovirus vectors contains at least one TR at the 5′ and 3′ ends, flanking a heterologous nucleotide sequence of interest (such as the macrophage specific promoter operably linked to the nucleic acid molecule encoding TIPE2). The TR at one end of the substrate is non-resolvable, i.e., it cannot be resolved (nicked) by Rep protein. During replication, the inability of Rep protein to resolve one of the TRs will result in a stable intermediate with the two “monomers” covalently attached by the non-functional (i.e., non-resolvable) TR. The heterologous nucleotide sequence may be in either orientation with respect to the non-resolvable TR.

The term “flanked” is not intended to indicate that the sequences are necessarily contiguous. For example, in the example in the previous paragraph, there may be intervening sequences between the heterologous nucleotide sequence and the TR. A sequence that is “flanked” by two other elements, indicates that one element is located 5′ to the sequence and the other is located 3′ to the sequence; however, there may be intervening sequences therebetween.

According to this embodiment, the template for producing the duplexed parvovirus vDNA is about half of the size of the wild-type (wt) parvovirus genome (e.g., AAV) corresponding to the capsid into which the vDNA will be packaged. In some embodiments, the template is from about 40% to about 55% of wt, such as from about 45% to about 52% of wt. Thus, the duplexed vDNA produced from this template can have a total size that is approximately the size of the wild-type parvovirus genome (e.g., AAV) corresponding to the capsid into which the vDNA will be packaged, e.g., from about 80% to about 105% of wt. In the case of AAV, the AAV capsid disfavors packaging of vDNA that substantially deviate in size from the wt AAV genome. In the case of an AAV capsid, the template can be approximately 5.2 kb in size or less. In other embodiments, the template is greater than about 3.6, 3.8, 4.0, 4.2, or 4.4 kb in length and/or less than about 5.4, 5.2, 5.0 or 4.8 kb in length.

In some embodiments, the heterologous nucleotide sequence(s) is less than about 2.5 kb in length (such as less than about 2.4 kb, for example less than about 2.2 kb in length, or less than about 2.1 kb in length) to facilitate packaging of the duplexed template by the parvovirus (e.g., AAV) capsid. In another embodiment, the template itself is duplexed, i.e., is a dimeric self-complementary molecule. According to this embodiment, the template comprises a resolvable TR at either end. The template further comprises a centrally-located non-resolvable TR. In some embodiments, each half of the template on either side of the non-resolvable TR is approximately the same length. Each half of the template (i.e., between the resolvable and non-resolvable TR) comprises one or more heterologous nucleotide sequence(s) of interest. The heterologous nucleotide sequence(s) in each half of the molecule is flanked by a resolvable TR and the central non-resolvable TR.

The sequences in either half of the template are substantially complementary (i.e., at least about 90%, 95%, 98%, 99% nucleotide sequence complementarity or more), so that the replication products from the template may form double-stranded molecules due to base-pairing between the complementary sequences. In other words, the template is essentially an inverted repeat with the two halves joined by the non-resolvable TR.

In some non-limiting examples, the heterologous nucleotide sequence(s) in each half of the template are essentially completely self-complementary (i.e., contains an insignificant number of mis-matched bases, or even no mismatched bases). In additional non-limiting examples, the two halves of the nucleotide sequence are essentially completely self-complementary.

The TR(s) (resolvable and non-resolvable) can be AAV sequences, such as serotypes 1, 2, 3, 4, 5, 6, 7, 8, or 9. The term “terminal repeat” includes synthetic sequences that function as an AAV inverted terminal repeat, such as the “double-D sequence” as described in U.S. Pat. No. 5,478,745, incorporated by reference. Resolvable AAV TRs need not have a wild-type TR sequence (e.g., a wild-type sequence may be altered by insertion, deletion, truncation or missense mutations), as long as the TR mediates the desired functions, such as virus packaging, integration, and/or provirus rescue, and the like. In some embodiments, the TRs are from the same parvovirus, e.g., both TR sequences are from AAV6.

The viral Rep protein(s) used for producing the duplexed vectors are selected with consideration for the source of the viral TRs. For example, the AAV5 TR interacts more efficiently with the AAV5 Rep protein.

The genomic sequences of the various autonomous parvoviruses and the different serotypes of AAV, as well as the sequences of the TRs, capsid subunits, and Rep proteins are known in the art. Such sequences may be found in the literature or in public databases such as GENBANK®. See, e.g., GENBANK® Accession Numbers NC 002077, NC 001863, NC 001862, NC 001829, NC 001729, NC 001701, NC 001510, NC 001401, AF063497, U89790, AF043303, AF028705, AF028704, J02275, J01901, J02275, X01457, AF288061, AH009962, AY028226, AY028223, NC 001358, NC 001540; the disclosures of which are incorporated by references as available on Dec. 30, 2019. See also, e.g., Chiorini et al., (1999) J. Virology 73:1309; Xiao et al., (1999) J. Virology 73:3994; Muramatsu et al., (1996) Virology 221:208; PCT Publication Nos. WO 00/28061, WO 99/61601, WO 98/11244; and U.S. Pat. No. 6,156,303, all incorporated by reference herein.

The non-resolvable TR may be produced by any method known in the art. For example, insertion into the TR will displace the nicking site (i.e., trs) and result in a non-resolvable TR. The designation of the various regions or elements within the TR are known in the art. An illustration of the regions within the AAV TR is provided in Fields et al., Virology, volume 2, chapter 69, FIG. 5, 3d ed., Lippincott-Raven Publishers. The insertion can be made into the sequence of the terminal resolution site (trs). Alternatively, the insertion may be made at a site between the Rep Binding Element (RBE) within the A element and the trs in the D element. The core sequence of the AAV trs site is known in the art and has been described by Snyder et al., (1990) Cell 60:105; Snyder et al., (1993) J. Virology 67:6096; Brister & Muzyczka, (2000) J. Virology 74:7762; Brister & Muzyczka, (1999) J. Virology 73:9325 (the disclosures of which are hereby incorporated by reference in their entireties). For example, Brister & Muzyczka, (1999) J. Virology 73:9325 describes a core trs sequence of 3′-CCGGT/TG-5* in the D element. Snyder et al., (1993) J. Virology 67:6096 identified the minimum trs sequence as 3′-GGT/TGA-5′, which substantially overlaps the sequence identified by Brister & Muzyczka. In some embodiments, the insertion is in the region of the trs site. The insertion may be of any suitable length that will reduce or substantially eliminate (e.g., by 60%, 70%), 80%. 90%, 95% or greater) resolution of the TR. In some embodiments, the insertion is at least about 3, 4, 5, 6, 10, 15, 20 or 30 nucleotides or more. There are no particular upper limits to the size of the inserted sequence, as long as suitable levels of viral replication and packaging are achieved (e.g., the insertion can be as long as 50, 100, 200 or 500 nucleotides or longer).

In another embodiment, the TR may be rendered non-resolvable by deletion of the trs site. The deletions can extend 1, 3, 5, 8, 10, 15, 20, 30 nucleotides or more beyond the trs site, as long as the template retains the desired functions. In addition to the trs site, some or all of the D element may be deleted. Deletions may further extend into the A element, however those skilled in the art will appreciate that it may be advantageous to retain the RBE in the A element, e.g., to facilitate efficient packaging. Deletions into the A element may be 2, 3, 4, 5, 8, 10, or 15 nucleotides in length or more, as long as the non-resolvable TR retains any other desired functions. It is further preferred that some or all of the parvovirus (e.g., AAV) sequences going beyond the D element outside the TR sequence (e.g., to the right of the D element) be deleted to prevent gene conversion to correct the altered TR.

As still a further alternative, the sequence at the nicking site may be mutated so that resolution by Rep protein is reduced or substantially eliminated. For example, A and/or C bases may be substituted for G and/or T bases at or near the nicking site. The effects of substitutions at the terminal resolution site on Rep cleavage have been described by Brister & Muzyczka, (1999) J. Virology 73:9325 (the disclosure of which is hereby incorporated by reference). As a further alternative, nucleotide substitutions in the regions surrounding the nicking site, which have been postulated to form a stem-loop structure, may also be used to reduce Rep cleavage at the terminal resolution site.

Those skilled in the art will appreciate that the alterations in the non-resolvable TR may be selected so as to maintain desired functions, if any, of the altered TR (e.g., packaging, Rep recognition, site-specific integration, and the like). In some embodiments, the TR will be resistant to the process of gene conversion as described by Samulski et al., (1983) Cell 33:135. Gene conversion at the non-resolvable TR will restore the trs site, which will generate a resolvable TR and result in an increase in the frequency of monomeric replication products. Gene conversion results by homologous recombination between the resolvable TR and the altered TR.

One strategy to reduce gene conversion is to produce virus using a cell line (such as a, mammalian cell line) that is defective for DNA repair, as known in the art, as these cell lines will be impaired in their ability to correct the mutations introduced into the viral template. Alternatively, templates that have a substantially reduced rate of gene conversion can be generated by introducing a region of non-homology into the non-resolvable TR. Non-homology in the region surrounding the trs element between the non-resolvable TR and the unaltered TR on the template will reduce or even substantially eliminate gene conversion.

Any suitable insertion or deletion may be introduced into the non-resolvable TR to generate a region of non-homology, as long as gene conversion is reduced or substantially eliminated. Strategies that employ deletions to create non-homology are preferred. It is further preferred that the deletion does not unduly impair replication and packaging of the template. In the case of a deletion, the same deletion may suffice to impair resolution of the trs site as well as to reduce gene conversion. In some embodiments, gene conversion can be reduced by insertions into the non-resolvable TR or, alternatively, into the A element between the RBE and the trs site. The insertion can be at least about 3, 4, 5, 6, 10, 15, 20 or 30 nucleotides or more nucleotides in length. There is no particular upper limit to the size of the inserted sequence, which may be as long as 50, 100, 200 or 500 nucleotides or longer, however, it is preferred that the insertion does not unduly impair replication and packaging of the template.

In some embodiments, the non-resolvable TR may be a naturally-occurring TR (or altered form thereof) that is non-resolvable under the conditions used. For example, the non-resolvable TR may not be recognized by the Rep proteins used to produce the vDNA from the template. To illustrate, the non-resolvable TR may be an autonomous parvovirus sequence that is not recognized by AAV Rep proteins. As a yet further alternative, the non-resolvable sequence may be any inverted repeat sequence that forms a hairpin structure and cannot be cleaved by the Rep proteins.

In other embodiments, a half-genome size template may be used to produce a parvovirus particle carrying a duplexed vDNA, produced from a half-genome sized template, as described in Hirata & Russell, (2000) J. Virology 74:4612, which describes packaging of paired monomers and transient RF intermediates when AAV genomes were reduced to less than half-size of the wtAAV genome (<2.5 kb). These investigators found that monomeric genomes were the preferred substrate for gene correction by homologous recombination, and that duplexed genomes functioned less well than did monomeric genomes in this assay. This report did not investigate or suggest the use of duplexed genomes as vectors for gene delivery.

In some embodiments, the template will be approximately one-half of the size of the vDNA that can be packaged by the parvovirus capsid. For example, for an AAV capsid, the template can be approximately one-half of the wt AAV genome in length, as described above. The template (as described above) is replicated to produce a duplexed vector genome (vDNA), which is capable of forming a double-stranded DNA under appropriate conditions. The duplexed molecule is substantially self-complementary so as to be capable of forming a double-stranded viral DNA (i.e., at least 90%, 95%, 98%, 99%) nucleotide sequence complementarity or more). Base-pairing between individual nucleotide bases or polynucleotide sequences is well-understood in the art. In some embodiments, the duplexed parvovirus viral DNA is essentially completely self-complementary (i.e., contains no or an insignificant number of mis-matched bases). In particular, it is preferred that the heterologous nucleotide sequence(s) (e.g., the sequences to be transcribed by the cell) are essentially completely self-complementary.

In general, the duplexed parvoviruses may contain non-complementarity to the extent that expression of the heterologous nucleotide sequence(s) from the duplexed parvovirus vector is more efficient than from a corresponding monomeric vector.

The duplexed parvoviruses provide the host cell with a double-stranded molecule that addresses the need for the host cell to convert the single-stranded rAAV vDNA into a double-stranded DNA. The presence of any substantial regions of non-complementarity within the virion DNA, in particular, within the heterologous nucleotide sequence(s) will likely be recognized by the host cell, and will result in DNA repair mechanisms being recruited to correct the mismatched bases, thereby counteracting the advantageous characteristics of the duplexed parvovirus vectors, e.g., the vectors reduce or eliminate the need for the host cell to process the viral template.

The vectors disclosed herein, such as the adenovirus and AAV vectors, include a macrophage specific promoter operably linked to a nucleic acid encoding TIPE2. In some embodiments, the promoter is a CD68 promoter, such as the human CD68 promoter. Optionally, the CD68 enhancer is also included. The CD68 promoter is commercially available from Addgene (pcDNA3-CD68 promoter/enhancer, Plasmid #34837). The CD68 gene sequence can be obtained at GENBANK® on the internet (ncbi.nlm.nih.gov/gene/968, incorporated herein by reference.

An exemplary sequence of the pcDNA2-CD68 promoter and enhancer are provided below:

(SEQ ID NO: 5)
GACGGATCGGGAGATCCTAGCGTTTAAACTTAAGCTTGGTACCGAGCTCGGATCCACTAGTCCAGTGTGG
TGGAATTCTGCAGATATCAAACTGCCTGTTTGGGCTTCTCATTTCTTACCTCCCCTTCCCTCTCCCACCTGC
TACTGGGTGCATCTCTGCTCCCCCCTTCCCCAGCAGATGGTTACCTTTGGGCTGTTGCTTTCTTGTCACCAT
CTGAGTTCTCAGACGCTGGAAAGCCATGTTCTCGGCTCTGTGAATGACAATGCTGACTGGAGTGCTGCCC
CTCTGTAAAGGGCTGGGTGTGGATGGTCACAAGCCCCTCACATGCCTCAGCCAAGAGGAAGTAGTACAG
GGGTCAGCCCAGAGGTCCAGGGGAAAGGAGTGGAAACCGATTTCCCCACCAAGGGAGGGGCCTGTACCT
CAGCTGTTCCCATAGCTTACTTGCCACAACTGCCAAGCAAGTTTCGCTGAGTTTGACACATGGATCCCTGT
GGATCAACTGCCCTAGGACTCCGTTTGCACCCATGTGACACTGTTGACTTTGCCCTGACGAAGCAGGGCC
AACAGTCCCCTAACTTAATTACAAAAACTAATGACTAAGAGAGAGGTGGCTAGAGCTGAGGCCCCTGAG
TCAGGCTGTGGGTGGGATCATCTCCAGTACAGGAAGTGAGACTTTCATTTCCTCCTTTCCAAGAGAGGGC
TGAGGGAGCAGGGTTGAGCAACTGGTGCAGACAGCCTAGCTGGACTTTGGGTGAGGCGGTTCAGCCATA
TCGAATTCTGCTGGGGCTACTGGCAGGTAAGGAGGAAGGAGGCTGAGGGGAGGGGGCCCCTGGGAGGG
AGCCTGCCCTGGGTTGCTAACCATCTCCTCTCTGCCAAAAGCCCAGGGGACTCAGCGGCCGCTCGAGTCT
AGAGGGCCCGTTTAAACCCGCTGATCAGCCTCGACTGTGCCTTCTAGTTGCCAGCCATCTGTTGTTTGCCC
CTCCCCCGTGCCTTCCTTGACCCTGGAAGGTGCCACTCCCACTGTCCTTTCCTAATAAAATGAGGAAATTG
CATCGCATTGTCTGAGTAGGTGTCATTCTATTCTGGGGGGTGGGGTGGGGCAGGACAGCAAGGGGGAGG
ATTGGGAAGACAATAGCAGGCATGCTGGGGATGCGGTGGGCTCTATGGCTTCTGAGGCGGAAAGAACCA
GCTGGGGCTCTAGGGGGTATCCCCACGCGCCCTGTAGCGGCGCATTAAGCGCGGCGGGTGTGGTGGTTAC
GCGCAGCGTGACCGCTACACTTGCCAGCGCCCTAGCGCCCGCTCCTTTCGCTTTCTTCCCTTCCTTTCTCGC
CACGTTCGCCGGCTTTCCCCGTCAAGCTCTAAATCGGGGCATCCCTTTAGGGTTCCGATTTAGTGCTTTAC
GGCACCTCGACCCCAAAAAACTTGATTAGGGTGATGGTTCACGTAGTGGGCCATCGCCCTGATAGACGGT
TTTTCGCCCTTTGACGTTGGAGTCCACGTTCTTTAATAGTGGACTCTTGTTCCAAACTGGAACAACACTCA
ACCCTATCTCGGTCTATTCTTTTGATTTATAAGGGATTTTGGGGATTTCGGCCTATTGGTTAAAAAATGAG
CTGATTTAACAAAAATTTAACGCGAATTAATTCTGTGGAATGTGTGTCAGTTAGGGTGTGGAAAGTCCCC
AGGCTCCCCAGGCAGGCAGAAGTATGCAAAGCATGCATCTCAATTAGTCAGCAACCAGGTGTGGAAAGT
CCCCAGGCTCCCCAGCAGGCAGAAGTATGCAAAGCATGCATCTCAATTAGTCAGCAACCATAGTCCCGCC
CCTAACTCCGCCCATCCCGCCCCTAACTCCGCCCAGTTCCGCCCATTCTCCGCCCCATGGCTGACTAATTT
TTTTTATTTATGCAGAGGCCGAGGCCGCCTCTGCCTCTGAGCTATTCCAGAAGTAGTGAGGAGGCTTTTTT
GGAGGCCTAGGCTTTTGCAAAAAGCTCCCGGGAGCTTGTATATCCATTTTCGGATCTGATCAAGAGACAG
GATGAGGATCGTTTCGCATGATTGAACAAGATGGATTGCACGCAGGTTCTCCGGCCGCTTGGGTGGAGAG
GCTATTCGGCTATGACTGGGCACAACAGACAATCGGCTGCTCTGATGCCGCCGTGTTCCGGCTGTCAGCG
CAGGGGCGCCCGGTTCTTTTTGTCAAGACCGACCTGTCCGGTGCCCTGAATGAACTGCAGGACGAGGCAG
CGCGGCTATCGTGGCTGGCCACGACGGGCGTTCCTTGCGCAGCTGTGCTCGACGTTGTCACTGAAGCGGG
AAGGGACTGGCTGCTATTGGGCGAAGTGCCGGGGCAGGATCTCCTGTCATCTCACCTTGCTCCTGCCGAG
AAAGTATCCATCATGGCTGATGCAATGCGGCGGCTGCATACGCTTGATCCGGCTACCTGCCCATTCGACC
ACCAAGCGAAACATCGCATCGAGCGAGCACGTACTCGGATGGAAGCCGGTCTTGTCGATCAGGATGATC
TGGACGAAGAGCATCAGGGGCTCGCGCCAGCCGAACTGTTCGCCAGGCTCAAGGCGCGCATGCCCGACG
GCGAGGATCTCGTCGTGACCCATGGCGATGCCTGCTTGCCGAATATCATGGTGGAAAATGGCCGCTTTTC
TGGATTCATCGACTGTGGCCGGCTGGGTGTGGCGGACCGCTATCAGGACATAGCGTTGGCTACCCGTGAT
ATTGCTGAAGAGCTTGGCGGCGAATGGGCTGACCGCTTCCTCGTGCTTTACGGTATCGCCGCTCCCGATTC
GCAGCGCATCGCCTTCTATCGCCTTCTTGACGAGTTCTTCTGAGCGGGACTCTGGGGTTCGAAATGACCG
ACCAAGCGACGCCCAACCTGCCATCACGAGATTTCGATTCCACCGCCGCCTTCTATGAAAGGTTGGGCTT
CGGAATCGTTTTCCGGGACGCCGGCTGGATGATCCTCCAGCGCGGGGATCTCATGCTGGAGTTCTTCGCC
CACCCCAACTTGTTTATTGCAGCTTATAATGGTTACAAATAAAGCAATAGCATCACAAATTTCACAAATA
AAGCATTTTTTTCACTGCATTCTAGTTGTGGTTTGTCCAAACTCATCAATGTATCTTATCATGTCTGTATAC
CGTCGACCTCTAGCTAGAGCTTGGCGTAATCATGGTCATAGCTGTTTCCTGTGTGAAATTGTTATCCGCTC
ACAATTCCACACAACATACGAGCCGGAAGCATAAAGTGTAAAGCCTGGGGTGCCTAATGAGTGAGCTAA
CTCACATTAATTGCGTTGCGCTCACTGCCCGCTTTCCAGTCGGGAAACCTGTCGTGCCAGCTGCATTAATG
AATCGGCCAACGCGCGGGGAGAGGCGGTTTGCGTATTGGGCGCTCTTCCGCTTCCTCGCTCACTGACTCG
CTGCGCTCGGTCGTTCGGCTGCGGCGAGCGGTATCAGCTCACTCAAAGGCGGTAATACGGTTATCCACAG
AATCAGGGGATAACGCAGGAAAGAACATGTGAGCAAAAGGCCAGCAAAAGGCCAGGAACCGTAAAAAG
GCCGCGTTGCTGGCGTTTTTCCATAGGCTCCGCCCCCCTGACGAGCATCACAAAAATCGACGCTCAAGTC
AGAGGTGGCGAAACCCGACAGGACTATAAAGATACCAGGCGTTTCCCCCTGGAAGCTCCCTCGTGCGCTC
TCCTGTTCCGACCCTGCCGCTTACCGGATACCTGTCCGCCTTTCTCCCTTCGGGAAGCGTGGCGCTTTCTC
AATGCTCACGCTGTAGGTATCTCAGTTCGGTGTAGGTCGTTCGCTCCAAGCTGGGCTGTGTGCACGAACC
CCCCGTTCAGCCCGACCGCTGCGCCTTATCCGGTAACTATCGTCTTGAGTCCAACCCGGTAAGACACGAC
TTATCGCCACTGGCAGCAGCCACTGGTAACAGGATTAGCAGAGCGAGGTATGTAGGCGGTGCTACAGAG
TTCTTGAAGTGGTGGCCTAACTACGGCTACACTAGAAGGACAGTATTTGGTATCTGCGCTCTGCTGAAGC
CAGTTACCTTCGGAAAAAGAGTTGGTAGCTCTTGATCCGGCAAACAAACCACCGCTGGTAGCGGTGGTTT
TTTTGTTTGCAAGCAGCAGATTACGCGCAGAAAAAAAGGATCTCAAGAAGATCCTTTGATCTTTTCTACG
GGGTCTGACGCTCAGTGGAACGAAAACTCACGTTAAGGGATTTTGGTCATGAGATTATCAAAAAGGATCT
TCACCTAGATCCTTTTAAATTAAAAATGAAGTTTTAAATCAATCTAAAGTATATATGAGTAAACTTGGTCT
GACAGTTACCAATGCTTAATCAGTGAGGCACCTATCTCAGCGATCTGTCTATTTCGTTCATCCATAGTTGC
CTGACTCCCCGTCGTGTAGATAACTACGATACGGGAGGGCTTACCATCTGGCCCCAGTGCTGCAATGATA
CCGCGAGACCCACGCTCACCGGCTCCAGATTTATCAGCAATAAACCAGCCAGCCGGAAGGGCCGAGCGC
AGAAGTGGTCCTGCAACTTTATCCGCCTCCATCCAGTCTATTAATTGTTGCCGGGAAGCTAGAGTAAGTA
GTTCGCCAGTTAATAGTTTGCGCAACGTTGTTGCCATTGCTACAGGCATCGTGGTGTCACGCTCGTCGTTT
GGTATGGCTTCATTCAGCTCCGGTTCCCAACGATCAAGGCGAGTTACATGATCCCCCATGTTGTGCAAAA
AAGCGGTTAGCTCCTTCGGTCCTCCGATCGTTGTCAGAAGTAAGTTGGCCGCAGTGTTATCACTCATGGTT
ATGGCAGCACTGCATAATTCTCTTACTGTCATGCCATCCGTAAGATGCTTTTCTGTGACTGGTGAGTACTC
AACCAAGTCATTCTGAGAATAGTGTATGCGGCGACCGAGTTGCTCTTGCCCGGCGTCAATACGGGATAAT
ACCGCGCCACATAGCAGAACTTTAAAAGTGCTCATCATTGGAAAACGTTCTTCGGGGCGAAAACTCTCAA
GGATCTTACCGCTGTTGAGATCCAGTTCGATGTAACCCACTCGTGCACCCAACTGATCTTCAGCATCTTTT
ACTTTCACCAGCGTTTCTGGGTGAGCAAAAACAGGAAGGCAAAATGCCGCAAAAAAGGGAATAAGGGCG
ACACGGAAATGTTGAATACTCATACTCTTCCTTTTTCAATATTATTGAAGCATTTATCAGGGTTATTGTCT
CATGAGCGGATACATATTTGAATGTATTTAGAAAAATAAACAAATAGGGGTTCCGCGCACATTTCCCCGA
AAAGTGCCACCTGACGTCGACGGATCGGGAGATCTCCCGATCCCCTATGGTCGACTCTCAGTACAATCTG
CTCTGATGCCGCATAGTTAAGCCA

As disclosed in Lang et al. (J Immunol. 2002 Apr. 1; 168(7):3402-11, incorporated herein by reference, the 2940 bp of sequence 5′ to the ATG and the 83-bp first intron of the human CD68 gene can be used. Optionally, the 83 base pair first intron can also be included. Additional information on the CD68 promoter is available in Greaves et al., Genomics 54: 165, incorporated herein by reference. In some embodiments, the CD68 promoter/enhancer −680/+140 is used, which includes both the promoter and part of the proximal enhancer.

In other embodiments, the macrophage specific promoter is the CD11b promoter. The CD11b promoter is commercially available from Addgene (in pGEM3zf(−), Plasmid #26168. The CD11b gene sequence can be obtained at GENBANK® on the internet (ncbi.nlm.nih.gov/gene/3684, incorporated herein by reference.

An exemplary CD11b promoter is provided below:

(SEQ ID NO: 6)
AGCTTGCATGCCTGCAGGTCGACTCTAGAGTCGACCTGCAGGCATGCAAGTTTTTTTTTTTTTTTAGAGAT
AAGAGTCTTGCTCTGTCGCCTAGGCTGGAGTGCAGTGGCACAATCTCTGCTCACTGCAACCTCCGCCTCC
AGGGTTCAAGTGATTCTGCTGCCTCAGCCTCCCAGGTGGGATTACAGGTGCCTGCCACCACGCCTGGCTA
ATTTTTTTGTCTTTTTAGTAAAGATGAGGTTTCACCATGTTGGGCAGGCTGGTTTCAATTGCTGACCTCAA
GTGAGCCACCCCGCCTCAGCCTCCCAAAATGCTAGGATTACAGGCATGAGCCACCGCACCCAGCCAAGTT
TGTACATATATTTTTGACTACACTTCTTAACTATTCTTAGGATAAATTACTAGAAGTGAAAATTCTTGGGT
GAAGAGCTTGAGGCCTTTACACACACACACACACACACACACACACACACACAAATAGGCTGGATGCAG
TGGCTCACACCTGTAATCTCAGCAGTTTGGGAGGCTGAGGAAGGAGGATCACTTGAGTCCAGGAGGTTG
AGAATAGCCTGAACAACATAGCAAGATCTTGTCTCTACAAAAAATTTAAAAAAAATTAGCTGGCCATGG
CAGCATGTGCCTGTAGTACCAGCTACTCGGAAGGCTGAGGTAGGAGGATCGCTTGAGCCCAGGAGGTTG
ATTGAAGCTGCAGTGAGCTGTGATTACACCACTGCACTCCAGCCTGGGCAACAGAGCTAGACTCTGTCTC
TAAAAAAAGCACAAAATAATATTTAAAAAGCACCAGGTATGCCTGTACTTGAGTTGTCTTTGTTGATGGC
TACAAATGAGGACAGCTCTGGCTGAAGGGCGCTTCCATTTCCATGGGCTGAAGGAGGGACATTTTGCAAA
GTGTGTTTTCAGGAAGACACAGAGTTTTACCTCCTACACTTGTTTGATCTGTATTAATGTTTGCTTATTTAT
TTATTTAATTTTTTTTTTGAGACAGAGTCTCACTCTGTCACCTGGGCTGGAGTGCAGTGGCATTATTGAGG
CTCATTGCAGTCTCAGACTCCTGAGCTCAAACAATCCTCCTGCCTCAGCCTCTGGAGTAGCTAGGACTAC
AGGCATGTGCCACCATGCCTGGCTAATTTTTTAAATGTATTTTTTTGTAGAGTCGGGGTCTCCCTATGTTG
CCCAGGCTGGAGTGCAGTGGTGTGATCCTAGCTCACTGCAGCCTGGACCTCGGGCTCAAGTAATTCTCAC
ACCTCAGCCTGTCCAGTAGCAGGGGCTACAGGCGCGCACCACCATGCCCAGCTAATTAAAAATATTTTTT
TGTAGAGACAGGGTCTCTCTATGTTGCCCAGGCTGGTTTCAAACTCCCAGGCTCAAGCAATCCTCCTGCCT
TGGCCTCCCAAAGTGCTGGCATTACAGGCGTGAGCCACTGCGCCTGGCCCGTATTAATGTTTAGAACACG
AATTCCAGGAGGCAGGCTAAGTCTATTCAGCTTGTTCATATGCTTGGGCCAACCCAAGAAACAAGTGGGT
GACAAATGGCACCTTTTGGATAGTGGTATTGACTTTGAAAGTTTGGGTCAGGAAGCTGGGGAGGAAGGG
TGGGCAGGCTGTGGGCAGTCCTGGGCGGAAGACCAGGCAGGGCTATGTGCTCACTGAGCCTCCGCCCTCT
TCCTTTGAATCTCTGATAGACTTCTGCCTCCTACTTCTCCTTTTCTGCCCTTCTTTGCTTTGGTGGCTTCCTT
GTGGTTCCTCAGTGGTGCCTGCAACCCCTGGTTCACCTCCTTCCAGGTTCTGGCTCCTTCCAGCCCGGGTA
CCGAGCTCGAATTCGCCCTATAGTGAGTCGTATTACAATTCACTGGCCGTCGTTTTACAACGTCGTGACTG
GGAAAACCCTGGCGTTACCCAACTTAATCGCCTTGCAGCACATCCCCCTTTCGCCAGCTGGCGTAATAGC
GAAGAGGCCCGCACCGATCGCCCTTCCCAACAGTTGCGCAGCCTGAATGGCGAATGGAAATTGTAAGCG
TTAATATTTTGTTAAAATTCGCGTTAAATTTTTGTTAAATCAGCTCATTTTTTAACCAATAGGCCGAAATC
GGCAAAATCCCTTATAAATCAAAAGAATAGACCGAGATAGGGTTGAGTGTTGTTCCAGTTTGGAACAAG
AGTCCACTATTAAAGAACGTGGACTCCAACGTCAAAGGGCGAAAAACCGTCTATCAGGGCGATGGCCCA
CTACGTGAACCATCACCCTAATCAAGTTTTTTGGGGTCGAGGTGCCGTAAAGCACTAAATCGGAACCCTA
AAGGGAGCCCCCGATTTAGAGCTTGACGGGGAAAGCCGGCGAACGTGGCGAGAAAGGAAGGGAAGAAA
GCGAAAGGAGCGGGCGCTAGGGCGCTGGCAAGTGTAGCGGTCACGCTGCGCGTAACCACCACACCCGCC
GCGCTTAATGCGCCGCTACAGGGCGCGTCCTGATGCGGTATTTTCTCCTTACGCATCTGTGCGGTATTTCA
CACCGCATATGGTGCACTCTCAGTACAATCTGCTCTGATGCCGCATAGTTAAGCCAGCCCCGACACCCGC
CAACACCCGCTGACGCGCCCTGACGGGCTTGTCTGCTCCCGGCATCCGCTTACAGACAAGCTGTGACCGT
CTCCGGGAGCTGCATGTGTCAGAGGTTTTCACCGTCATCACCGAAACGCGCGAGACGAAAGGGCCTCGTG
ATACGCCTATTTTTATAGGTTAATGTCATGATAATAATGGTTTCTTAGACGTCAGGTGGCACTTTTCGGGG
AAATGTGCGCGGAACCCCTATTTGTTTATTTTTCTAAATACATTCAAATATGTATCCGCTCATGAGACAAT
AACCCTGATAAATGCTTCAATAATATTGAAAAAGGAAGAGTATGAGTATTCAACATTTCCGTGTCGCCCT
TATTCCCTTTTTTGCGGCATTTTGCCTTCCTGTTTTTGCTCACCCAGAAACGCTGGTGAAAGTAAAAGATG
CTGAAGATCAGTTGGGTGCACGAGTGGGTTACATCGAACTGGATCTCAACAGCGGTAAGATCCTTGAGA
GTTTTCGCCCCGAAGAACGTTTTCCAATGATGAGCACTTTTAAAGTTCTGCTATGTGGCGCGGTATTATCC
CGTATTGACGCCGGGCAAGAGCAACTCGGTCGCCGCATACACTATTCTCAGAATGACTTGGTTGAGTACT
CACCAGTCACAGAAAAGCATCTTACGGATGGCATGACAGTAAGAGAATTATGCAGTGCTGCCATAACCA
TGAGTGATAACACTGCGGCCAACTTACTTCTGACAACGATCGGAGGACCGAAGGAGCTAACCGCTTTTTT
GCACAACATGGGGGATCATGTAACTCGCCTTGATCGTTGGGAACCGGAGCTGAATGAAGCCATACCAAA
CGACGAGCGTGACACCACGATGCCTGTAGCAATGGCAACAACGTTGCGCAAACTATTAACTGGCGAACT
ACTTACTCTAGCTTCCCGGCAACAATTAATAGACTGGATGGAGGCGGATAAAGTTGCAGGACCACTTCTG
CGCTCGGCCCTTCCGGCTGGCTGGTTTATTGCTGATAAATCTGGAGCCGGTGAGCGTGGGTCTCGCGGTA
TCATTGCAGCACTGGGGCCAGATGGTAAGCCCTCCCGTATCGTAGTTATCTACACGACGGGGAGTCAGGC
AACTATGGATGAACGAAATAGACAGATCGCTGAGATAGGTGCCTCACTGATTAAGCATTGGTAACTGTCA
GACCAAGTTTACTCATATATACTTTAGATTGATTTAAAACTTCATTTTTAATTTAAAAGGATCTAGGTGAA
GATCCTTTTTGATAATCTCATGACCAAAATCCCTTAACGTGAGTTTTCGTTCCACTGAGCGTCAGACCCCG
TAGAAAAGATCAAAGGATCTTCTTGAGATCCTTTTTTTCTGCGCGTAATCTGCTGCTTGCAAACAAAAAA
ACCACCGCTACCAGCGGTGGTTTGTTTGCCGGATCAAGAGCTACCAACTCTTTTTCCGAAGGTAACTGGC
TTCAGCAGAGCGCAGATACCAAATACTGTTCTTCTAGTGTAGCCGTAGTTAGGCCACCACTTCAAGAACT
CTGTAGCACCGCCTACATACCTCGCTCTGCTAATCCTGTTACCAGTGGCTGCTGCCAGTGGCGATAAGTCG
TGTCTTACCGGGTTGGACTCAAGACGATAGTTACCGGATAAGGCGCAGCGGTCGGGCTGAACGGGGGGT
TCGTGCACACAGCCCAGCTTGGAGCGAACGACCTACACCGAACTGAGATACCTACAGCGTGAGCTATGA
GAAAGCGCCACGCTTCCCGAAGGGAGAAAGGCGGACAGGTATCCGGTAAGCGGCAGGGTCGGAACAGG
AGAGCGCACGAGGGAGCTTCCAGGGGGAAACGCCTGGTATCTTTATAGTCCTGTCGGGTTTCGCCACCTC
TGACTTGAGCGTCGATTTTTGTGATGCTCGTCAGGGGGGCGGAGCCTATGGAAAAACGCCAGCAACGCG
GCCTTTTTACGGTTCCTGGCCTTTTGCTGGCCTTTTGCTCACATGTTCTTTCCTGCGTTATCCCCTGATTCTG
TGGATAACCGTATTACCGCCTTTGAGTGAGCTGATACCGCTCGCCGCAGCCGAACGACCGAGCGCAGCGA
GTCAGTGAGCGAGGAAGCGGAAGAGCGCCCAATACGCAAACCGCCTCTCCCCGCGCGTTGGCCGATTCA
TTAATGCAGCTGGCACGACAGGTTTCCCGACTGGAAAGCGGGCAGTGAGCGCAACGCAATTAATGTGAG
TTAGCTCACTCATTAGGCACCCCAGGCTTTACACTTTATGCTTCCGGCTCGTATGTTGTGTGGAATTGTGA
GCGGATAACAATTTCACACAGGAAACAGCTATGACCATGATTACGCCAAGCTATTTAGGTGACACTATAG
AATACTCA

The CD11b promoter directs high-level expression of reporter genes in macrophages in transgenic mice, see Dziennis set al., Blood. 1995 Jan. 15. 85(2):319-29, incorporated herein by reference.

Other regulatory elements of use include with viral enhancers and other macrophage specific promoters. One of skill in the art will readily appreciate that variants of a promoter can be used, such as promoters at least 95%, 96%, 97%, 98%, 99% identical to the CD68 or CD11b promoter, that still provide the promoter functions, such that a heterologous nucleic acid operably linked to the promoter is expressed in macrophages. In additional embodiments, the promoter can include at most 100, 95, 90, 85, 80, 75, 70, 65, 60, 55, 50, 45, 40, 35, 30, 25, 20, or 15 nucleic acid substitutions, provided the promoter functions, such that a heterologous nucleic acid operably linked to the promoter can be expressed when transferred into a macrophage. In more embodiments, the promoter can include at most 15, 14, 13, 12, 11, 10, 9, 8, 7, 6, 5, 4, 3, 2, 1 nucleic acid substitutions, provided the promoter functions, such that a heterologous nucleic acid operably linked to the promoter can be expressed when transferred into a macrophage. Additional nucleotides can be added, provided the promoter functions, such that a heterologous nucleic acid operably linked to the promoter is expressed when transferred into a in a macrophage.

In some embodiments, a promoter can be used that is at least 95%, 96%, 97%, 98%, 99% identical to SEQ ID NO: 5 or SEQ ID NO: 6, that still provides the promoter functions, such that a heterologous nucleic acid operably linked to the promoter is expressed in macrophages. In additional embodiments, the promoter can include at most 100, 95, 90, 85, 80, 75, 70, 65, 60, 55, 50, 45, 40, 35, 30, 25, 20, or 15 nucleic acid substitutions in SEQ ID NO: 5 or SEQ ID NO: 6, provided the promoter functions, such that a heterologous nucleic acid operably linked to the promoter can be expressed when transferred into a macrophage. In more embodiments, the promoter can include at most 15, 14, 13, 12, 11, 10, 9, 8, 7, 6, 5, 4, 3, 2, 1 nucleic acid substitutions in SEQ ID NO: 5 or SEQ ID NO: 6, provided the promoter functions, such that a heterologous nucleic acid operably linked to the promoter can be expressed when transferred into a macrophage. Additional nucleotides can be added, provided the promoter functions, such that a heterologous nucleic acid operably linked to the promoter is expressed when transferred into a in a macrophage.

The macrophage specific promoter is operably linked to a heterologous nucleic acid encoding TIPE2. An exemplary human TIPE protein is provided below:

(SEQ ID NO: 1)
MESFSSKSLALQAEKKLLSKMAGRSVAHLFIDETSSEVLDELYRVSKEYT
HSRPQAQRVIKDLIKVAIKVAVLHRNGSFGPSELALATRFRQKLRQGAMT
ALSFGEVDFTFEAAVLAGLLTECRDVLLELVEHHLTPKSHGRIRHVFDHF
SDPGLLTALYGPDFTQHLGKICDGLRKLLDEGKL.

An exemplary mouse TIPE protein is provided below:

(SEQ ID NO: 2)
MESFSSKSLALQAEKKLLSKMAGRSVAHLFIDETSSEVLDELYRVSKEYT
HSRPKAQRVIKDLIKVAVKVAVLHRSGCFGPGELALATRFRQKLRQGAMT
ALSFGEVDFTFEAAVLAGLLVECRDILLELVEHHLTPKSHDRIRHVFDHY
SDPDLLAALYGPDFTQHLGKICDGLRKLLDEGKL

In some embodiments, vectors of use in the disclosed methods encode an amino acid sequence at least about 95%, 96%, 97%, 98% or 99% identical to SEQ ID NO: 1 or SEQ ID NO: 2, wherein the protein functions as a TIPE2 protein. In more embodiments, vectors of use in the disclosed method encode SEQ ID NO: 1 or SEQ ID NO: 2 with at most 1, 2, 3, 4, 5, 6, 7, 8, 9, or 10 conservative amino acid substitutions. In other embodiments, vectors of use in the disclosed method encode SEQ ID NO: 1 or SEQ ID NO: 2 with at most 1, 2, 3, 4, or 5 conservative amino acid substitutions.

Without being bound by theory, the central region of TIPE2 was initially thought to constitute a DED (death effector) domain. However, 3D-structure data reveal a previously uncharacterized fold that is different from the predicted fold of a DED domain. It consists of a large, hydrophobic central cavity that is poised for cofactor binding (by similarity). Thus, in specific non-limiting examples, substitutions are made outside of this hydrophobic central cavity. In other specific non-limiting examples, substitutions are made outside of the domain that is similar to the DED domain, which is

RSVAHLFIDETSSEVLDELYRVSKEYTHSRPQAQRVIKDLIKVAIKVAVL
HRNGSFGPSELALATRFRQKLRQGAMTALSFGEVDFTFEAAVLAGLLTEC
RDVLLELVEHHLTPKSHGRIRHVFDHFSDPGLLTALYGPDFTQHLGKICD
GLRKLLDEGKL (amino acids 24-181 of SEQ ID NO: 1)

Thus, in some embodiments, the amino acid sequence is at least about 95%, 96%, 97%, 98% or 99% identical to SEQ ID NO: 1, and wherein there are no amino acid substitutions at residues 24-181 of SEQ ID NO: 1. In other embodiment, the amino sequence includes at most 1, 2, 3, 4 or 5 conservative amino acid substitutions in SEQ ID NO: 1, wherein there are no amino acid substitutions at residues 24-181 of SEQ ID NO: 1.

An exemplary nucleic acid molecule encoding SEQ ID NO: 1 is provided below:

(SEQ ID NO: 3)
ATGGAGTCCTTCAGCTCAAAGAGCCTGGCACTGCAAGCAGAGAAGAAGCT
ACTGAGTAAGATGGCGGGTCGCTCTGTGGCTCATCTCTTCATAGATGAGA
CAAGCAGTGAGGTGCTAGATGAGCTCTACCGTGTGTCCAAGGAGTACACG
CACAGCCGGCCCCAGGCCCAGCGCGTGATCAAGGACCTGATCAAAGTGGC
CATCAAGGTGGCTGTGCTGCACCGCAATGGCTCCTTTGGCCCCAGTGAGC
TGGCCCTGGCTACCCGCTTTCGCCAGAAGCTGCGGCAGGGTGCCATGACG
GCACTTAGCTTTGGTGAGGTAGACTTCACCTTCGAGGCTGCTGTTCTGGC
TGGCCTGCTGACCGAGTGCCGGGATGTGCTGCTAGAGTTGGTGGAACACC
ACCTCACGCCCAAGTCACATGGCCGCATCCGCCACGTGTTTGATCACTTC
TCTGACCCAGGTCTGCTCACGGCCCTCTATGGGCCTGACTTCACTCAGCA
CCTTGGCAAGATCTGTGACGGACTCAGGAAGCTGCTAGACGAAGGGAAGC
TCTGA

An exemplary nucleic acid sequence encoding SEQ ID NO: 2 is set for the below:

(SEQ ID NO: 4)
ATGGAGTCCTTCAGCTCAAAGAGTCTGGCACTACAAGCGGAGAAGAAGCT
GCTGAGTAAAATGGCTGGTCGGTCCGTGGCGCATCTCTTTATCGACGAGA
ACCAGCAGCGAGGTGCTGACGAGCTTTACCGCGTGTCCAAAGAATACACG
CACAGCCGGCCCAAGGCACAGCGGGTGATCAAAGACCTCATCAAGGTAGC
GGTTAAAGTGGCTGTGCTGCACCGCAGTGGCTGCTTTGGCCCTGGGGAGC
TGGCTCTGGCTACACGATTTCGTCAGAAGCTACGGCAGGGCGCCATGACC
GCACTTAGCTTCGGTGAGGTGGACTTCACCTTTGAGGCTGCCGTGCTAGC
AGGTCTGCTCGTCGAGTGCCGGGACATTCTGCTGGAGCTGGTGGAGCACC
ACCTCACACCCAAGTCACATGACCGCATCAGGCACGTGTTTGATCACTAC
TCTGACCCCGACCTGCTGGCTGCCCTCTATGGGCCTGACTTCACTCAGCA
CCTTGGCAAGATCTGTGATGGGCTCCGGAAGCTGCTGGACGAGGGCAAGC
TCTGA.

Using the genetic code, one of skill in the art can readily produce other nucleic acid molecules that encode a TIPE2 protein. Human TIPE sequences are disclosed for example in GENBANK® Accession No. NM_024575.5 (nucleotide) and GENBANK® Accession No. NP_078851.2 (protein), both incorporated by reference as available on Dec. 31, 2019. Mouse TIPE sequences are disclosed for example in GENBANK® Accession No. NM_027206.3 (nucleotide) and GENBANK® Accession No. NP_081482.1 (protein), both incorporated by reference as available on Dec. 31, 2019. A nucleic acid molecule encoding a TIPE2 protein can be at least bout 95%, 96%, 97%, 98% or 99% identical to SEQ ID NO: 3 or SEQ ID NO: 4. In some embodiments, the nucleic acid molecule encodes an amino acid sequence is at least about 95%, 96%, 97%, 98% or 99% identical to SEQ ID NO: 1, wherein there are no amino acid substitutions at residues 24-181 of SEQ ID NO: 1.

In some embodiments, a vector of use includes a gene encoding a selectable marker, which includes, but are not limited to, a protein whose expression can be readily detected such as a fluorescent or luminescent protein or an enzyme that acts on a substrate to produce a colored, fluorescent, or luminescent substance (“detectable markers”). There are other genes of use, such as genes that encode drug resistance of provide a function that can be used to purify cells. Selectable markers include neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also selectable makers.

### Pharmaceutical Compositions and Methods of Use

Methods are provided for increasing macrophage polarization to M2 macrophages. These methods include administering to the subject a vector, such as an adenovirus vector or an AAV vector, including a macrophage specific promoter operably linked to a nucleic acid sequence encoding a TIPE2 protein. In some embodiments, the vector is administered locally to an organ of the subject. The organ can be any organ of interest, including the eye, joints, liver, kidneys, heart, skin, gastrointestinal tract (mouth, esophagus, small intestine, large intestine, colon, etc.), an organ of the respiratory system (lungs, trachea, etc.), an organ of endocrine system (pituitary, pancreas, etc.) an organ of the reproductive system (ovaries, uterus, penis, testicles, etc.), a bone, or any other organ of interest, such as, but not limited to a muscle, tendon, thyroid, adrenal, bladder, lymph node, spleen, brain, adipose tissue, blood vessels, spinal cord or a nerve. In some non-limiting examples, the organ is the pancreas.

In some embodiments, methods are provided for polarizing macrophages to become M2 macrophages in the pancreas of a subject. These methods include administering to the subject a vector including a macrophage specific promoter operably linked to a nucleic acid sequence encoding a TIPE2 protein to the pancreas. In some embodiments, the vector is administered intraductally into a pancreatic duct of the subject.

Methods are also provided for treating a subject with T1D. These methods include administering to the subject a vector, such as an adenovirus vector or an AAV vector, including a macrophage specific promoter operably linked to a nucleic acid sequence encoding a TIPE2 protein to the pancreas of the subject. In some embodiments, the vector is administered intraductally into a pancreatic duct of the subject.

For in vivo delivery, a vector, such as an adenovirus or an AAV vector can be formulated into a pharmaceutical composition and will generally be administered locally or systemically. In some embodiments, for use in subjects with diabetes, the vector is administered directly to the pancreas. In other embodiments, intraductally into a pancreatic duct of the subject. In other embodiments, the subject has diabetes, such as T1D.

The subject can be any mammalian subject, including human and veterinary subjects. The subject can be a child or an adult. The method can include selecting a subject of interest, such as a subject with diabetes. The subject can also be administered insulin. The method can include polarizing macrophages to become M2 macrophages in the pancreas of a diabetic subject. In some embodiments, the vector is administered intraductally.

In some examples, a subject with diabetes may be clinically diagnosed by a fasting plasma glucose (FPG) concentration of greater than or equal to 7.0 millimole per liter (mmol/L) (126 milligram per deciliter (mg/dL)), or a plasma glucose concentration of greater than or equal to 11.1 mmol/L (200 mg/dL) at about two hours after an oral glucose tolerance test (OGTT) with a 75 gram (g) load, or in a patient with classic symptoms of hyperglycemia or hyperglycemic crisis, a random plasma glucose concentration of greater than or equal to 11.1 mmol/L (200 mg/dL), or HbA1c levels of greater than or equal to 6.5%. In other examples, a subject with pre-diabetes may be diagnosed by impaired glucose tolerance (IGT). An OGTT two-hour plasma glucose of greater than or equal to 140 mg/dL and less than 200 mg/dL (7.8-11.0 mM), or a fasting plasma glucose (FPG) concentration of greater than or equal to 100 mg/dL and less than 125 mg/dL (5.6-6.9 mmol/L), or HbA1c levels of greater than or equal to 5.7% and less than 6.4% (5.7-6.4%) is considered to be IGT, and indicates that a subject has pre-diabetes. Additional information can be found in Standards of Medical Care in Diabetes—2010 (American Diabetes Association, Diabetes Care 33:S11-61, 2010, incorporated herein by reference).

In some embodiments, the subject can have new onset diabetes. Thus, in some embodiments, the subject can have diabetes for at most about 1, 2, 3, 4, 5, 6, 7, 8, 9, or days, at most about 1, 2, 3, 4, 5, 6, 7, or 8 weeks, or at most about 1, 2, 3, 4, 5 or 6 months. In some embodiments, the subject has new onset diabetes, which is the initial detection of the diabetic condition.

In some embodiments, a subject is selected for treatment that has T1D. The subject can be a pediatric subject. The subject can be an adult subject.

Without being bound by theory, the disclosed methods preserve pancreatic beta cells in a subject. Generally, these cells produce insulin. In some embodiments, the subject is a subject with T1D has a reduced auto-immune response to the pancreatic beta cells. In some embodiments, T cell and/or B cells do not produce an immune response to the pancreatic beta cells produced by the disclosed methods. Thus, in some embodiments, the subject does not mount an autoimmune response to the pancreatic beta cells. In specific non-limiting examples, the subject has reduced destruction of the pancreatic beta cells and does not exhibit an increased lymphocyte invasion of the islets. In some embodiments, macrophages in the pancreas of the subject are polarized to become M2 macrophages. In specific non-limiting examples, M2 macrophages are about 0.05% to about 100%, such as about 0.05% to 50%, such as about 1% to about 50%, or about 0.05%, 1%, 5%, 10%, 15%, 20%, 25%, 30%, 35%, 40%, 45% or 50% of the total macrophages. In other embodiments, the absolute number of M2 macrophages increases by about 100 fold, 200 fold, 300 fold, 400 fold or 500 fold, such as more than about 300 fold, such as about 300 fold to about 500 fold.

Appropriate doses depend on the subject being treated (e.g., human or nonhuman primate or other mammal), age and general condition of the subject to be treated, the severity of the condition being treated, the mode of administration of the AAV vector/virion, among other factors. An appropriate effective amount can be readily determined by one of skill in the art. Thus, a “therapeutically effective amount” will fall in a relatively broad range that can be determined through clinical trials. The method can include measuring an outcome, such as insulin production, improvement in a fasting plasma glucose tolerance test, or the number of M2 macrophages.

The disclosed methods can include administering other therapeutic agents, such as insulin. The disclosed methods can also include having the subject make lifestyle modifications.

In some embodiments, the subject is also administered a viral vector encoding pancreas duodenal homeobox protein (Pdx)1 and Musculoaponeurotic fibrosarcoma oncogene homolog A MafA, and optionally encoding Neurogenin (Ngn) 3, to induce the production of beta cells in the pancreas. This vector can be an AAV or adenoviral vector, and can be administered intraductally. a viral vector, such as an adenoviral vector or an adeno-associated viral vector encoding Pdx1 and MafA can be infused through the pancreatic duct of a subject, such as a subject with T1D, in order to reprogram alpha-cells into functional beta-cells. These beta cells are immunologically unrecognized for an extended period by the immune system of the subject. The viral vector can be delivered to the subject using endoscopic retrograde cholangiopancreatography (ERCP). In some embodiments, the subject is not administered Ngn3 or a nucleic acid encoding Ngn3.

In specific non-limiting examples, the subject is administered a vector comprising a glucagon promoter operably linked to a nucleic acid sequence encoding heterologous Pdx1 and a nucleic acid sequence encoding MafA, wherein the vector does not encode Ngn3. In other embodiments, the subject is administered Ngn3 or a nucleic acid encoding Ngn3. In specific non-limiting examples, the subject is administered a vector comprising a glucagon promoter operably linked to a nucleic acid sequence encoding heterologous Pdx1 and a nucleic acid sequence encoding MafA and a nucleic acid sequence encoding Ngn3. Exemplary vectors of use, and methods for administering these vectors, are disclosed in U.S. Pat. No. 10,071,172, incorporated herein by reference.

For in vivo injection, i.e., injection directly to the subject, a therapeutically effective dose will be on the order of from about 10⁵ to 10¹⁶ of the AAV virions, such as 10⁸ to 10¹⁴ AAV virions. The dose, of course, depends on the efficiency of transduction, promoter strength, the stability of the message and the protein encoded thereby, and clinical factors. Effective dosages can be readily established by one of ordinary skill in the art through routine trials establishing dose response curves.

Dosage treatment may be a single dose schedule or a multiple dose schedule to ultimately deliver the amount specified above. Moreover, the subject may be administered as many doses as appropriate. Thus, the subject may be given, e.g., 10⁵ to 10¹⁶ AAV virions in a single dose, or two, four, five, six or more doses that collectively result in delivery of, e.g., 10⁵ to 10¹⁶ AAV virions. One of skill in the art can readily determine an appropriate number of doses to administer.

In some embodiments, the AAV is administered at a dose of about 1×10¹¹ to about 1×10¹⁴ viral particles (vp)/kg. In some examples, the AAV is administered at a dose of about 1×10¹² to about 8×10¹³ vp/kg. In other examples, the AAV is administered at a dose of about 1×10¹³ to about 6×10¹³ vp/kg. In specific non-limiting examples, the AAV is administered at a dose of at least about 1×10¹¹, at least about 5×10¹¹, at least about 1×10¹², at least about 5×10¹², at least about 1×10¹³, at least about 5×10¹³, or at least about 1×10¹⁴ vp/kg. In other non-limiting examples, the rAAV is administered at a dose of no more than about 5×10¹¹, no more than about 1×10¹², no more than about 5×10¹², no more than about 1×10¹³, no more than about 5×10¹³, or no more than about 1×10¹⁴ vp/kg. In one non-limiting example, the AAV is administered at a dose of about 1×1012 vp/kg. The AAV can be administered in a single dose, or in multiple doses (such as 2, 3, 4, 5, 6, 7, 8, 9 or 10 doses) as needed for the desired therapeutic results, such as the polarization of macrophages to M2 macrophages and/or treatment of T1D.

Pharmaceutical compositions include sufficient genetic material to produce a therapeutically effective amount of TIPE2. In some embodiments, AAV virions will be present in the subject compositions in an amount sufficient to provide a therapeutic effect, such as the production of M2 macrophages and/or the treatment of diabetes, such as T1D, when given in one or more doses.

AAV virions can be provided as lyophilized preparations and diluted in a stabilizing compositions for immediate or future use. Alternatively, the AAV virions can be provided immediately after production and stored for future use.

The pharmaceutical compositions can contain the vector, such as the rAAV vector, and/or virions, and a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in REMINGTON'S PHARMACEUTICAL SCIENCES (Mack Pub. Co., N.J. 1991).

In some embodiments, the excipients confer a protective effect on the AAV virion such that loss of AAV virions, as well as transduceability resulting from formulation procedures, packaging, storage, transport, and the like, is minimized. These excipient compositions are therefore considered “virion-stabilizing” in the sense that they provide higher AAV virion titers and higher transduceability levels than their non-protected counterparts, as measured using standard assays, see, for example, Published U.S. Application No. 2012/0219528, incorporated herein by reference. These Compositions therefore demonstrate “enhanced transduceability levels” as compared to compositions lacking the particular excipients described herein, and are therefore more stable than their non-protected counterparts.

Exemplary excipients that can used to protect the AAV virion from activity degradative conditions include, but are not limited to, detergents, proteins, e.g., ovalbumin and bovine serum albumin, amino acids, e.g., glycine, polyhydric and dihydric alcohols, such as but not limited to polyethylene glycols (PEG) of varying molecular weights, such as PEG-200, PEG-400, PEG-600, PEG-1000, PEG-1450, PEG-3350, PEG-6000, PEG-8000 and any molecular weights in between these values, with molecular weights of 1500 to 6000 preferred, propylene glycols (PG), sugar alcohols, such as a carbohydrate, for example, sorbitol. The detergent, when present, can be an anionic, a cationic, a zwitterionic or a nonionic detergent. An exemplary detergent is a nonionic detergent. One suitable type of nonionic detergent is a sorbitan ester, e.g., polyoxyethylenesorbitan monolaurate (TWEEN®-20) polyoxyethylenesorbitan monopalmitate (TWEEN®-40), polyoxyethylenesorbitan monostearate (TWEEN®-60), polyoxyethylenesorbitan tristearate (TWEEN®-65), polyoxyethylenesorbitan monooleate (TWEEN®-80), polyoxyethylenesorbitan trioleate (TWEEN®-85), such as TWEEN®-20 and/or TWEEN®-80. These excipients are commercially available from a number of vendors, such as Sigma, St. Louis, Mo.

The amount of the various excipients present in any of the disclosed compositions varies and is readily determined by one of skill in the art. For example, a protein excipient, such as BSA, if present, will can be present at a concentration of between 1.0 weight (wt.) % to about 20 wt. %, such as 10 wt. %. If an amino acid such as glycine is used in the formulations, it can be present at a concentration of about 1 wt. % to about 5 wt. %. A carbohydrate, such as sorbitol, if present, can be present at a concentration of about 0.1 wt % to about 10 wt. %, such as between about 0.5 wt. % to about 15 wt. %, or about 1 wt. % to about 5 wt. %. If polyethylene glycol is present, it can generally be present on the order of about 2 wt. % to about 40 wt. %, such as about 10 wt. % top about 25 wt. %. If propylene glycol is used in the subject formulations, it will typically be present at a concentration of about 2 wt. % to about 60 wt. %, such as about 5 wt. % to about 30 wt. %. I f a detergent such as a sorbitan ester (TWEEN®) is present, it can be present at a concentration of about 0.05 wt. % to about 5 wt. %, such as between about 0.1 wt. % and about 1 wt %, see U.S. Published Patent Application No. 2012/0219528, which is incorporated herein by reference. In one example, an aqueous virion-stabilizing formulation comprises a carbohydrate, such as sorbitol, at a concentration of between 0.1 wt. % to about 10 wt. %, such as between about 1 wt. % to about 5 wt. %, and a detergent, such as a sorbitan ester (TWEEN®) at a concentration of between about 0.05 wt. % and about 5 wt. %, such as between about 0.1 wt. % and about 1 wt. %. Virions are generally present in the composition in an amount sufficient to provide a therapeutic effect when given in one or more doses, as defined above.

The pharmaceutical compositions can include a contrast dye is administered in addition to the viral vector, such an adenoviral vector, including a macrophage-specific promoter operably lined to a nucleic acid molecule encoding TIPE2. The contrast dye can be a low-osmolar low-viscosity non-ionic dye, a low-viscosity high-osmolar dye, or a dissociable high-viscosity dye. In specific non-limiting examples, the dye is Iopromid, Ioglicinate, or Ioxaglinate. Thus, provided herein is a pharmaceutical composition including a) an adeno-associated virus vector, such as rAAV6, comprising a macrophage-specific promoter, such as a CD68 or CD11b promoter, operably linked to a nucleic acid molecule encoding TIPE2; b) a buffer; and c) a contrast dye for endoscopic retrograde cholangiopancreatography. Any of the AAV vectors disclosed herein can be included in this composition. The AAV vector can be encapsulated in a virion. The composition can be formulated for administration to the pancreatic duct. In some embodiments, an additional AAV vector of a same or different serotype, is also included. This additional AVV vector can encode Pdx1 and MafA. In some non-limiting examples, the additional AAV vector is an AAV8 vector. In further non-limiting examples, the additional AAV vector includes a glucagon promoter operably linked to a nucleic acid molecule encoding Pdx1 and MafA. In more non-limiting examples, the additional AAV vector does not encoded Ngn3. In yet other non-limiting examples, the additional AAV vector encodes Ngn3.

The disclosed pharmaceutical compositions including a viral vector, such an adenoviral vector, including a macrophage promoter operably linked to a nucleic acid molecule encoding TIPE, or a virion, can be delivered to humans or other animals by any means, including orally, intravenously, intramuscularly, intraperitoneally, intranasally, intradermally, intrathecally, subcutaneously, via inhalation or via suppository. In one non-limiting example, the composition is administered into the pancreatic duct of a subject in vivo.

One exemplary method for intraductal administration is Endoscopic Retrograde Cholangiopancreatography (ERCP). ERCP is an endoscopic technique that involves the placement of a side-viewing instrument (generally either an endoscope or duodenoscope) within the descending duodenum. The procedure eliminates the need for invasive surgical procedures for administration to the pancreatic duct.

In an ERCP procedure, the patient will generally lie on their side on an examining table. The patient will then be given medication to help numb the back of the patient's throat, and a sedative to help the patient relax during the examination. The patient then swallows the endoscope. The thin, flexible endoscope is passed carefully through the alimentary canal of the patient. The physician guides the endoscope through the patient's esophagus, stomach, and the first part of the small intestine known as the duodenum. Because of the endoscope's relatively small diameter, most patients can tolerate the unusualness of having the endoscope advanced through the opening of their mouth.

The physician stops the advancement of the endoscope when the endoscope reaches the junction where the ducts of the biliary tree and pancreas open into the duodenum. This location is called the papilla of Vater, or also commonly referred to as the ampulla of Vater. The papilla of Vater is a small mound of tissue looking and acting similarly to a nipple. The papilla of Vater emits a substance known as bile into the small intestine, as well as pancreatic secretions that contain digestive enzymes. Bile is a combination of chemicals made in the liver and is necessary in the act of digestion. Bile is stored and concentrated in the gallbladder between meals. When digestive indicators stimulate the gallbladder, however, the gallbladder squeezes the bile through the common bile duct and subsequently through the papilla of Vater. The pancreas secretes enzymes in response to a meal, and the enzymes help digest carbohydrates, fats, and proteins.

The patient will be instructed (or manually maneuvered) to lie flat on their stomach once the endoscope reaches the papilla of Vater. For visualization or treatment specifically within the biliary tree, the distal end of the endoscope is positioned proximate the papilla of Vater. A catheter is then advanced through the endoscope until the distal tip of the catheter emerges from the opening at the endoscope's distal end. The distal end of the catheter is guided through the endoscope's orifice to the papilla of Vater (located between the sphincter of Oddi) and advanced beyond the common channel and into the common bile duct. In the case of pancreas-specific delivery of reagents, the pancreatic duct proper can be entered.

ERCP catheters can be constructed from Teflon, polyurethane and polyaminde ERCP catheters also can also be constructed from resin comprised of nylon and PEBA (see U.S. Pat. No. 5,843,028), and can be constructed for use by a single operator (see U.S. Pat. No. 7,179,252). At times, a spring wire guide may be placed in the lumen of the catheter to assist in cannulation of the ducts. A stylet, used to stiffen the catheter, must first be removed prior to spring wire guide insertion. An inflatable balloon tip catheter may be used to prevent back flow out of the targeted ductal system.

A dual or multi-lumen ERCP catheter in which one lumen could be utilized to accommodate the spring wire guide or diagnostic or therapeutic device, and in which a second lumen could be utilized for contrast media and/or dye infusion and or for administration of a pharmaceutical composition including a viral vector, such an adenoviral vector. In some embodiments, a contrast dye is administered in addition to the pharmaceutical composition including a viral vector, such an adenoviral vector, or an adeno-associated virus vector, such as rAAV6, comprising a macrophage-specific promoter, such as a CD68 or CD11b promoter, operably linked to a nucleic acid molecule encoding TIPE2MafA. The contrast dye can be a low-osmolar low-viscosity non-ionic dye, a low-viscosity high-osmolar dye, or a dissociable high-viscosity dye. In specific non-limiting examples, the dye is Iopromid, Ioglicinate, or Ioxaglinate. Endoscopes have been designed for the delivery of more than one liquid solution, such as a first liquid composition including a viral vector, such an adenoviral vector, oe an adeno-associated virus vector, such as rAAV6, comprising a macrophage-specific promoter, such as a CD68 or CD11b promoter, operably linked to a nucleic acid molecule encoding TIPE2, and a second liquid composition including dye, see U.S. Pat. No. 7,597,662, which is incorporated herein by reference. Thus, the pharmaceutical composition including the viral vector and the dye can be delivered in the same or separate liquid compositions. Methods and devices for using biliary catheters for accessing the biliary tree for ERCP procedures are disclosed in U.S. Pat. Nos. 5,843,028, 5,397,302 5,320,602, which are incorporated by reference herein.

In additional examples, the vector is administered using a viral infusion technique into a pancreatic duct. Suitable methods are disclosed, for example, in Guo et al. Laboratory Invest. 93: 1241-1253, 2013, incorporated by reference herein.

### EXAMPLES

A sustained immune attack on the insulin-producing pancreatic beta-cells characterizes T1D. In the current study, an adeno-associated virus (AAV) vector was generated that carried TNF-alpha-induced protein 8-like 2 (TIPE2) under a macrophage-specific CD68 promoter (AAV-pCD68-TIPE2). This construct successfully induced M2 polarization of pancreatic macrophages in vitro, as well as in vivo, when the virus was given via a pancreatic intraductal infusion. A single ductal infusion of AAV-pCD68-TIPE2 reversed the progression of diabetes in NOD mice, a mouse model for human T1D. Mechanistically, AAV-pCD68-TIPE2 triggered upregulation of complement receptor of the immunoglobulin family (CRIg) in macrophages and increased Foxp3+ regulatory T-cells (Tregs) in the NOD mouse pancreas, which were both necessary for the AAV-pCD68-TIPE2-induced diabetes reversal in NOD mice. Collectively, the data demonstrated a clinically translatable approach to treat autoimmune T1D through the induction of M2-like macrophage polarization.

### Example 1

### Altered Intrapancreatic Polarization of Macrophages Reverses Autoimmune Diabetes

The expression of TIPE2 was forced specifically in the macrophages within the autoimmune NOD mouse pancreas in order to convert M1-like macrophages, which make up the vast majority of diabetic macrophages, into M2-like macrophages. First, TIPE2 levels were examined in normal, FACS-purified F4/80+CD206− M1 macrophages and F4/80+CD206+ M2 macrophages from mouse pancreas (FIG. 1A). It was found that M2 macrophages expressed significantly higher levels of TIPE2 than M1 macrophages by RT-qPCR (FIG. 1B), and by Western blot (FIG. 1C). Next, in order to specifically induce expression of TIPE2 in the macrophages within the pancreas, an AAV serotype 6 was generated carrying TIPE2 and a GFP reporter under a CD68 promoter (simplified as AAV-pCD68-TIPE2). The CD68 promoter restricts transgene expression to macrophages when infused in vivo. An AAV serotype 6 carrying a GFP reporter alone under a CD68 promoter (simplified as AAV-pCD68-GFP) was also generated as a control to exclude possible confounding effects of viral infection itself (FIG. 1D). FACS-sorted pancreatic F4/80+CD206− M1 macrophages were transduced with AAV-pCD68-TIPE2 or AAV-pCD68-GFP. TIPE2 was successfully induced in these in vitro F4/80+CD206− M1 macrophages as shown by Western blot (FIG. 1E), which was associated with downregulation of M1-associated factors, including iNOS, TNFα, IL-6, IL-1β, IL-12, and was also associated with upregulation of M2-associated factors, including IL-10, CD163, CD206, CD301, arginase 1 (ARG1), Fizz1 and Ym1 (FIG. 1F). Moreover, by immunostaining ARG1 protein was significantly induced in the transduced (GFP+) cells (FIG. 1G). Together, these data suggest that forced expression of TIPE2 induces a general alteration of the expression profile of M1 macrophages toward an M2-like polarization, rather than just activating certain M2-associated genes or simply causing apoptosis of M1 macrophages.

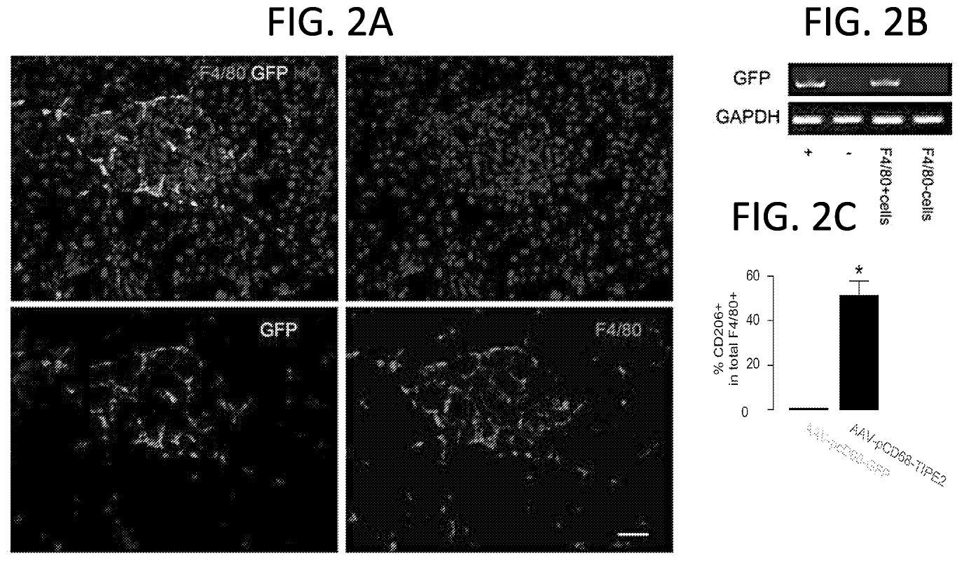

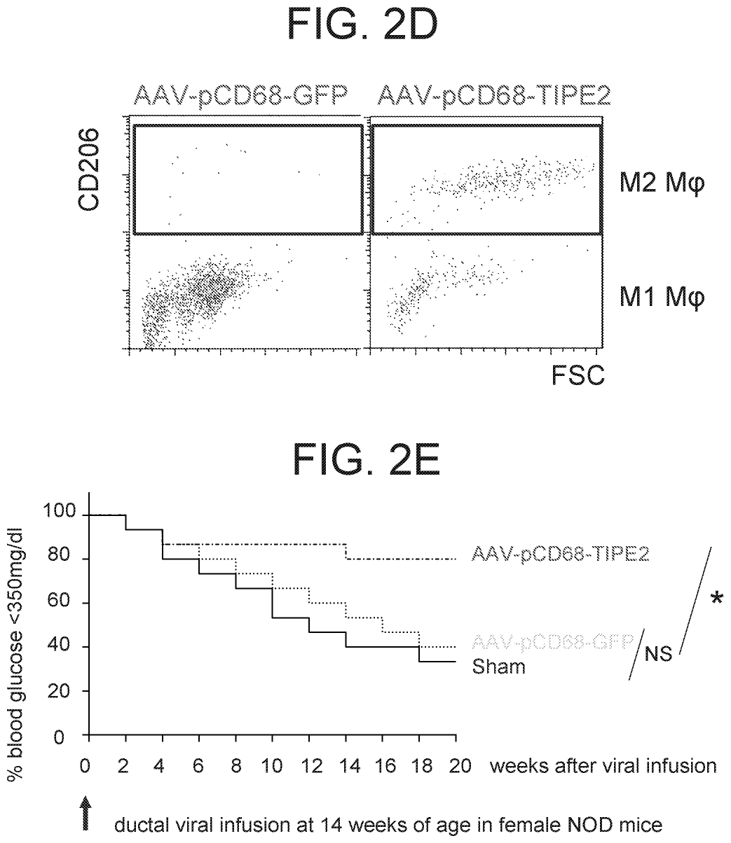

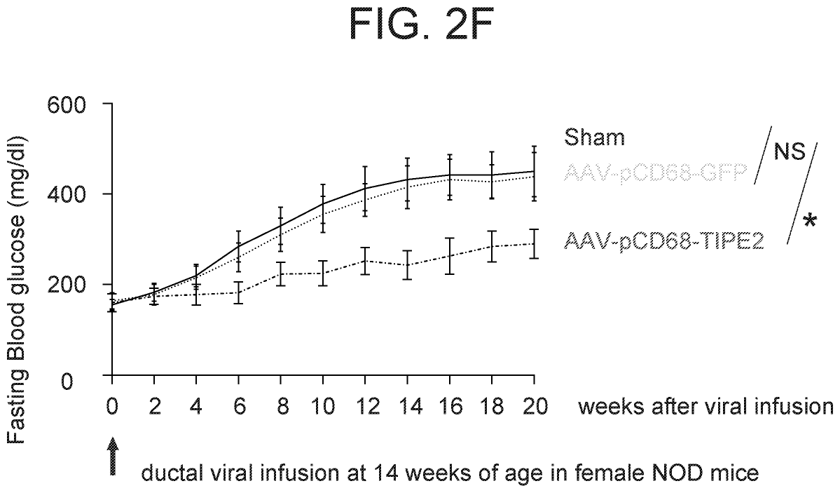

Next, AAV-pCD68-TIPE2 or control AAV-pCD68-GFP viruses (1012 genome copy particles in 120 μl volume) were introduced into the mouse pancreas via intra-pancreatic ductal infusion in 14-week old female NOD mice that were selected based on a fasting blood glucose between 150 and 200 mg/dl. This glycemic range was selected since many mice with lower glycemic levels at this age do not develop diabetes, while mice with higher glycemic level may become very ill due to glucose or lipid toxicity (Xiao et al., Autoimmune Diabetes. Cell Stem Cell 22, 78-90 e74 (2018); Xiao et al., J Biol Chem 292, 3456-3465 (2017); Xiao et al., Proc Natl Acad Sci USA 111, E1211-1220 (2014); Xiao et al., Nat Protoc 9, 2719-2724 (2014); Xiao et al., J Biol Chem 288, 25297-25308 (2013); Xiao et al., Diabetologia 57, 991-1000 (2014)). First, the specificity of the CD68 promoter for macrophages in vivo was assessed by immunohistochemistry. GFP signal was detected exclusively in F4/80+ macrophages in the AAV-pCD68-TIPE2-infused NOD mouse pancreas at day 7 after viral infusion (FIG. 2A). Moreover, both the macrophages inside islets and those in the inter-acinar stroma were transduced (FIG. 2A). In order to exclude the possibility that a minor non-macrophage population in the pancreas may express CD68, and thus potentially cause an off-target effect, GFP transcripts were checked in FAC-sorted F4/80+ and F4/80− pancreatic cells, which represent macrophages and non-macrophages in the pancreas, respectively. GFP transcripts were highly detected in FAC-sorted F4/80+ pancreatic cells, but not in F4/80− pancreatic cells, suggesting that the CD68 promoter in the AAV-pCD68-TIPE2 and control AAV-pCD68-GFP vectors specifically drove the expression of transgene expression in only macrophages in the mouse pancreas (FIG. 2B). Furthermore, in vivo TIPE2-induced M2 macrophage polarization was confirmed by a significant increase in the percentage of CD206+ M2 macrophages out of all F4/80+ pancreatic macrophages by flow cytometry (FIGS. 2C-D). On the other hand, the total number of F4/80+ macrophages did not significantly change, suggesting that polarization of M1 to M2, rather than induced cell death of M1 macrophages, contributed to this alteration in macrophage subpopulations. Fasting blood glucose after ductal infusion with AAV-pCD68-GFP or AAV-pCD68-TIPE2 in the NOD mice was monitored. The AAV-pCD68-TIPE2 infusion led to an M2 macrophage polarization, and actually reversed the onset and progression of diabetes (“diabetes” defined as fasting blood glucose >350 mg/dl) in NOD mice (FIG. 2E). In addition, the baseline fasting blood glucose levels were significantly lower in the NOD mice that had received AAV-pCD68-TIPE2 compared with those that had received AAV-pCD68-GFP (FIG. 2F). Thus, TIPE2-induced M2-macrophage polarization in pancreas appears to reverse new-onset diabetes in NOD mice.

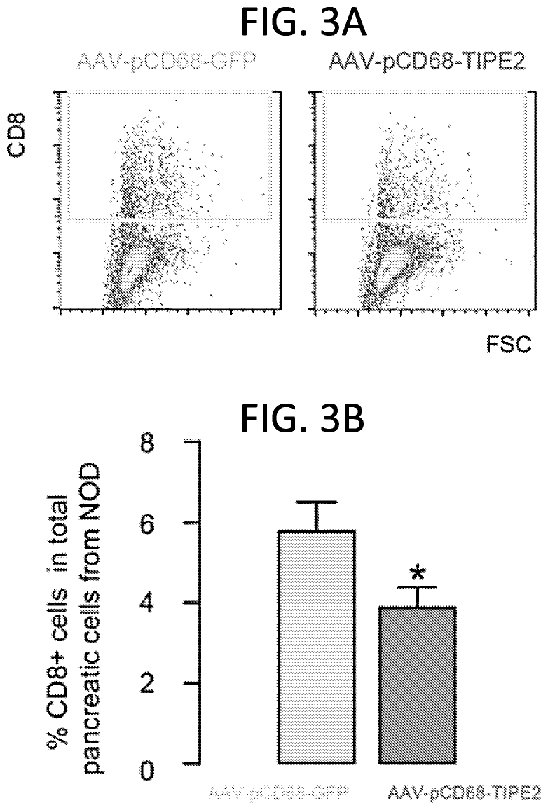

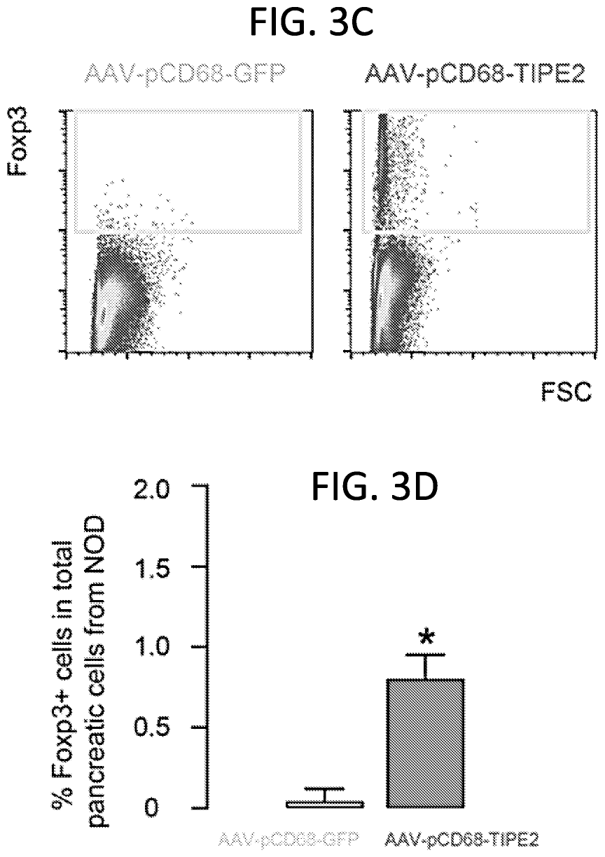

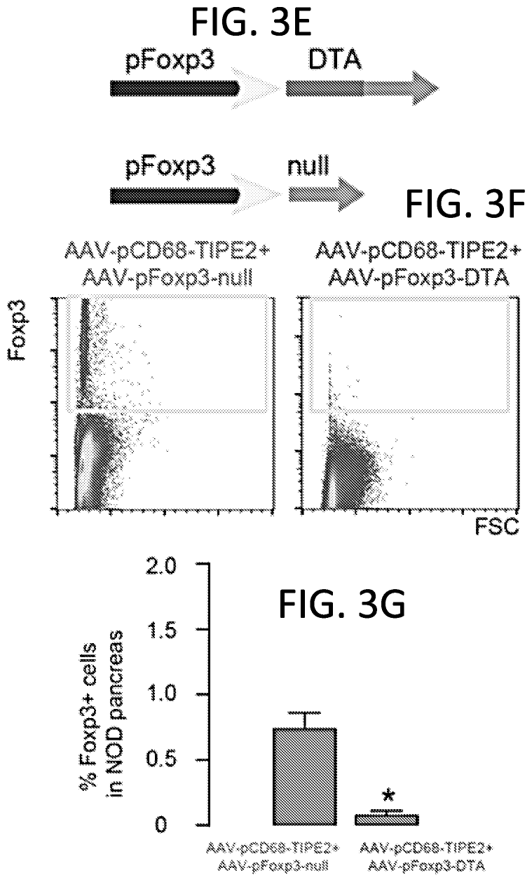

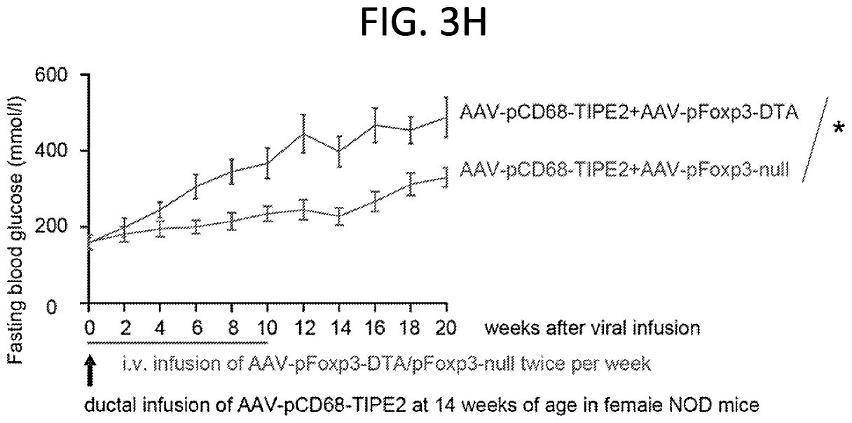

In order to understand the underlying mechanism of diabetes reversal, the number of cytotoxic T-cells and regulatory T-cells (Treg) were examined in the AAV-pCD68-TIPE2-treated NOD mouse pancreas, compared to the control AAV-pCD68-GFP-treated pancreas, by flow cytometry. CD8 is a marker for cytotoxic T-cells, which are the major effector T-cells that mediate the autoimmune attack on beta-cells in NOD mice. Foxp3 is the best validated marker for Tregs, which can suppress autoimmunity in NOD mice. Foxp3 has been shown to be necessary and sufficient to induce Treg differentiation from immature CD4+ T-cells (22). A significant decrease in CD8+ cytotoxic T-cells was detected in the mouse pancreas, shown by representative FACS plots (FIG. 3A) and by quantification (FIG. 3B), and a significant increase in Foxp3+ Tregs after AAV-pCD68-TIPE2 infusion (FIGS. 3C-D). These alterations in T-cell subtypes likely contribute to the reversal of diabetes through reduced autoimmunity in the NOD mouse pancreas after AAV-pCD68-TIPE2 treatment. Since the effect of TIPE2 on Tregs was quite dramatic, compared to the more modest changes in CD8+ cytotoxic T-cells, the subsequent studies below focused on Tregs.

In order to examine whether the significant increase in Foxp3+ Tregs induced by AAV-pCD68-TIPE2 may be directly responsible for the improved diabetic phenotype in NOD mice, an AAV serotype 6 carrying diphtheria toxin A (DTA) under a Treg-specific Foxp3 promoter was generated. The control AAV vector was a null construct under the Foxp3 promoter. Both viruses did not carry a fluorescent reporter so as to be distinguishable from TIPE2 viruses when co-administrated (FIG. 3E). Twice-per-week tail vein injections of 1010 genome copy particles of AAV-pFoxp3-DTA or control AAV-pFoxp3-null were given in 50 μl saline into NOD mice that had received a prior intra-ductal infusion of AAV-TIPE2. AAV-pFoxp3-DTA virus, but not control virus, significantly decreased the Foxp3+ Tregs in the mouse pancreas at one week after viral injection (FIGS. 3F-G), and completely abolished the ameliorating effects of AAV-pCD68-TIPE2 on diabetes in NOD mice (FIG. 3H). Here, a repetitive systemic administration of AAV-pFoxp3-DT and AAV-pFoxp3-null was used instead of a single intrapancreatic ductal infusion, because pancreatic T-cells would likely be readily replenished from the circulation.

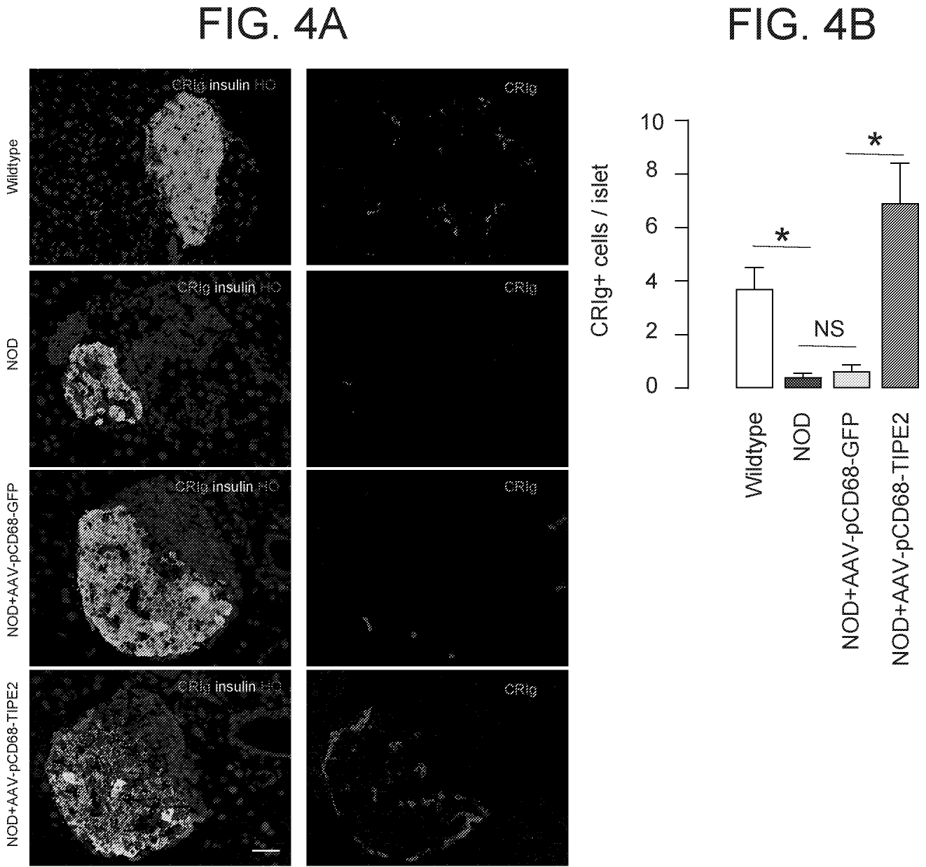

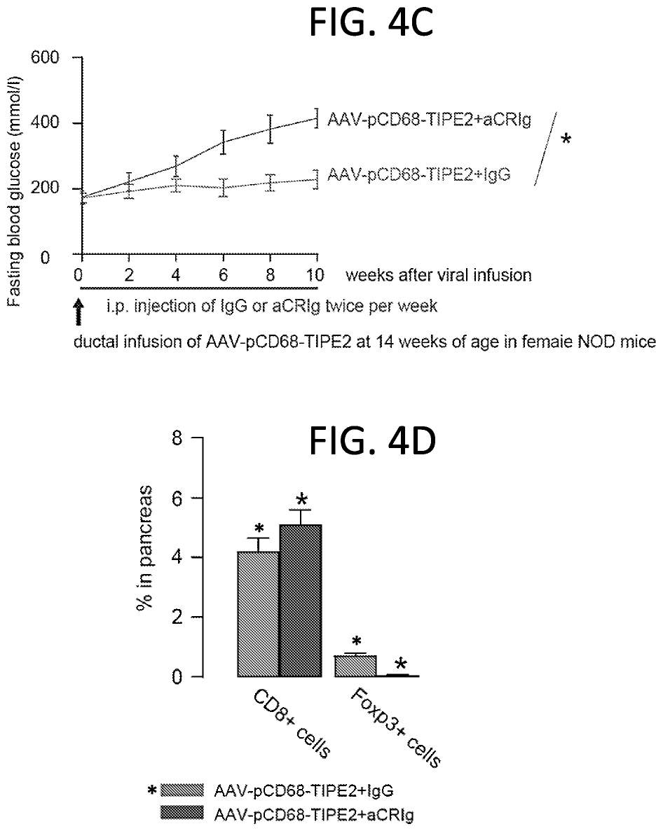

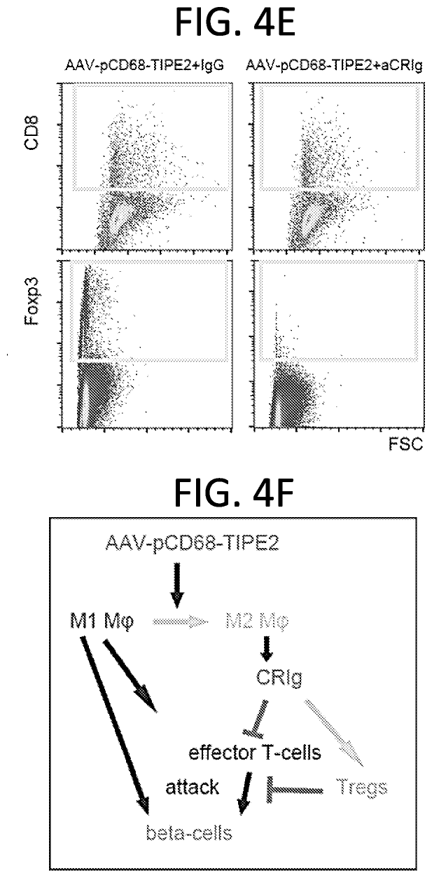

The effects of macrophages on T-cell differentiation and proliferation have been extensively reported (Jun, et al. J Exp Med 189, 347-358 (1999)). However, a direct effect of M2 macrophage polarization on Tregs has not been solidly shown. Complement receptor of the immunoglobulin family (CRIg) has been shown to be specifically expressed in tissue-resident macrophages of the mouse pancreas, and CRIg expression is thought to promote immunological tolerance through suppressing effector T-cells and through activating Tregs (Yuan, et al., eLife 6, (2017); Fu, et al., Nat Immunol 13, 361-368 (2012)). Since the data showed a significant reduction in the number of effector cytotoxic T-cells and a dramatic increase in the number of Tregs by TIPE2 expression in macrophages, it was hypothesized that CRIg may be a mediator of these effects. CRIg staining was performed on wild-type mouse pancreas, and on NOD mouse pancreas prior to viral treatment, and on NOD mouse pancreas 7 days after ductal infusion with AAV-pCD68-TIPE2 or AAV-pCD68-GFP. Some CRIg+ cells were detected in wild-type mouse islets, but significantly fewer were detected in the NOD mouse pancreas prior to viral treatment, or 7 days after infusion with AAV-pCD68-GFP. However, significantly higher numbers of CRIg+ cells were detected in the NOD mouse pancreas 7 days after infusion with AAV-pCD68-TIPE2, suggesting that TIPE2-induced M2 polarization may increase CRIg levels in these tissue-resident macrophages (FIGS. 4A-B).

The increase in the number of Tregs after TIPE2 treatment appeared to be much more pronounced than the decrease in cytotoxic T-cells. The modest reduction in number of cytotoxic T-cells in the NOD mouse pancreas may be due to suppression of the proliferation of effector cytotoxic T-cells without directly inducing cell loss through apoptosis or senescence. This suppression of proliferation of effector cytotoxic T-cells by TIPE2 may be mediated by CRIg, since it was shown that TIPE2 induces CRIg in macrophages, and it is known that CRIg inhibits T-cell proliferation through the T cell receptor (TCR) (Yuan, et al., eLife 6, (2017); Fu, et al., Nat Immunol 13, 361-368 (2012)). Since TCR is much more highly expressed on cytotoxic T-cells than on Tregs, CRIg would have been expected to inhibit proliferation of cytotoxic T-cells rather than Tregs (23, 24). On the other hand, CRIg has been shown to promote Treg differentiation and also stabilize the differentiated Tregs (Yuan, et al., eLife 6, (2017); Fu, et al., Nat Immunol 13, 361-368 (2012)). Therefore, the dramatic increase in the number of Tregs by TIPE2 treatment may also be mediated through CRIg.

CRIg+ tissue-resident macrophages have been shown to form a protective barrier surrounding pancreatic islets to regulate adaptive immunity and immune tolerance (Yuan, et al., eLife 6, (2017); Fu, et al., Nat Immunol 13, 361-368 (2012)). Without being bound by theory, TIPE2-induced M2 polarization of tissue-resident macrophages may create long-term immunosuppression in the NOD mouse pancreas through CRIg-mediated suppression of effector cytotoxic T-cells and CRIg-mediated activation of Tregs. To test this possibility, an i.p. injection of neutralizing antibody against CRIg was introduced into AAV-pCD68-TIPE2-treated NOD mice twice per week. This treatment abolished the effects of TIPE2 on both blood glucose (FIG. 4C), and the changes in number of effector T-cells and Tregs (FIGS. 4D-E). CRIg is exclusively expressed by tissue-resident macrophages, which are primarily maintained by self-replication rather than by replenishment from circulating monocytes (Carrero et al., Proc Natl Acad Sci USA 114, E10418-E10427 (2017).). Thus, the single pancreatic ductal infusion of AAV-pCD68-TIPE2 likely led to persistent alteration of the phenotype of tissue-resident macrophages, and subsequently long-term immunosuppression in NOD mice. The data indicate the importance of tissue-resident macrophages as a target for the therapy of T1D, since effector cells like cytotoxic T-cells have a high turn-over rate, and may be much more difficult to steadily target over the long run.

Furthermore, the expression of CRIg was investigated in a limited number of human pancreatic specimens. Surprisingly, many CRIg+ cells were found in non-diabetic pancreatic specimens, but very little in the diabetic specimens, suggesting that CRIg may also play a critical role in autoimmune diabetes in humans.

Collectively, the results show that TIPE2-triggered M2 polarization of tissue-resident macrophages induces upregulation of CRIg, which subsequently reverses diabetes progression in the NOD mouse through suppression of effector cytotoxic T-cells and activation of Tregs, as illustrated (FIG. 4E). Intra-ductal infusion of AAV carrying Pdx1 and MafA can reprogram alpha-cells into insulin-producing beta-like cells that reverse diabetes in NOD mice for 4 months, after which the blood glucose increased again, likely due to a return of autoimmunity. Here, a treatment is provided, using a similar administration technique, to suppress autoimmunity in NOD mice. Intriguingly, these two approaches could be applied together in one treatment to address the two critical issues in T1D therapy, namely restoration of functional beta-cell mass and suppression of autoimmunity. The combined application of these two strategies is clinically translatable to humans, given the availability of intraductal infusion in humans through endoscopic retrograde cholangiopancreatography (ERCP), and given that AAVs have been widely used in gene therapy clinical trials (Mandel and Burger, Curr Opin Mol Ther 6, 482-490 (2004): Wells, Mol Ther 25, 834-835 (2017)).

### Example 2

### Methods

Specimens and mouse manipulation: All mouse experiments were approved and were carried out in accordance with the approved guidelines. Human pancreatic specimens were obtained with obtained informed content. Female C57BL/6 and NOD mice were all purchased from the Jackson Lab (Bar Harbor, Me., USA). C57BL/6 mice were used at 10 weeks of age. Female NOD mice were used when the blood glucose reached a specified level. Exclusion criteria: the only exclusions were NOD mice that failed to develop high blood glucose after 16 weeks of age. Randomization and blind assessment were used in all animal studies. Measurements of mouse blood glucose were performed at 10 am after a three-hour fasting period. Pancreatic intraductal viral infusion was performed as described previously (Xiao et al., Nat Protoc 9, 2719-2724 (2014)), in which 150 μl viruses [10¹² genome copy particle (GCP)/ml] were infused at a rate of 5 μl/min.

Virus production: AAV serotype 6 vectors were generated by transfection of human embryonic kidney 293 cells as described before (Guo et al., Journal of Virological Methods 183, 139-146 (2012); Guo et al., Bioengineered 4, (2012)). Human TIPE2 was cut down by NheI and XhoI from a commercial plasmid (AAV0700399) purchased from Applied Biological Materials Inc. (Richmond, BC, Canada). Human CD68 promoter was obtained from an Addgene plasmid (#34837, Watertown, Mass., USA) (Lang, et al., J Immunol 168, 3402-3411 (2002)). Human Foxp3 promoter was cloned by Mlu1 and BsrQ1 from genomic DNA from human embryonic kidney 293 cells. Transfection was performed with Lipofectamine 3000 reagent (Invitrogen, CA, Carlsbad, USA), according to the instructions of the manufacturer. Purification of AAV vectors were described before (Guo et al., Journal of Virological Methods 183, 139-146 (2012)), in which the empty capsid was removed from the sublayer formed after PEG-aqueous partitioning, without requirement for a density gradient. Most of the empty capsid was removed, and the remaining empty capsid was less than 19% (by TEM measurement) in the final purified virus solution. The prepared virus was stored at −80° C. Titration of viral vectors was determined using a dot-blot assay.

RNA isolation, quantitative polymerase chain reaction (RT-qPCR): RNA extraction and cDNA synthesis have been described before (Xiao et al., Cell Stem Cell 22, 78-90 e74 (2018)). RT-qPCR primers were all purchased from Qiagen (Valencia, Calif., USA). They were GAPDH (QT01658692), TIPE2 (QT02075962), iNOS (QT00100275), TNFα (QT00104006), IL-6 (QT00098875), IL-12 (QT01048334), IL-10 (QT00106169), CD163 (QT00123074), CD206 (QT00103012), CD301 (QT00151011), ARG1 (QT00134288), Fizz1 (QT00254359) and Ym1 (QT00108829). RT-qPCR was performed as described before (Xiao et al., Cell Stem Cell 22, 78-90 e74 (2018)). Values were normalized against GAPDH, which proved to be stable across the samples, and then compared to controls.

Flow cytometry: Digestion of the pancreas and flow cytometry analysis of pancreatic cells were done as described (Xiao et al., Proc Natl Acad Sci USA 111, E1211-1220 (2014)). Antibodies used in flow cytometry were APC-conjugated F4/80 (eBioscience), FITC-conjugated CD206, PEcy5-conjugated CD8 and PEcy7-conjugated Foxp3 (Becton-Dickinson Biosciences, San Jose, Calif., USA). The flow cytometry data were analyzed by Flowjo (version 11.0, Flowjo LLC, Ashland, Oreg., USA).

Immunocytochemistry, immunohistochemistry and Western blot: All the mice received heart perfusion to remove red blood cells from the vessels before the pancreas was harvested, as described before (Xiao et al., T-Cell Proliferation. Diabetes 62, 1217-1226 (2013)). Pancreas samples were then fixed in zinc (BD Biosciences) for 6 hours before an additional 2 hours fixation in 4% formalin, then cryo-protected in 30% sucrose overnight, followed by freezing in a longitudinal orientation (from tail to head of the pancreas) and sectioned at 6 μm. GFP was detected by direct fluorescence. Western blot was performed as described before (Xiao et al., Endocrinology, en20151986 (2016)). Primary antibodies are: guinea pig polyclonal insulin-specific (Dako, Carpinteria, Calif., USA), rabbit polyclonal CRIg-specific, ARG1-specific and CD45-specific (Abcam, Cambridge, Mass., USA), rabbit polyclonal MafA-specific (Bethyl Laboratories, Inc., Montgomery, USA), rat F4/80-specific (Invitrogen). No antigen retrieval was necessary. Secondary antibodies for indirect fluorescent staining were Cy2, Cy3, or Cy5 conjugated rabbit-, rat-, and guinea pig-specific (Jackson ImmunoResearch Labs, West Grove, Pa., USA). Nuclear staining was performed with Hoechst solution (HO, Becton-Dickinson Biosciences, San Jose, Calif., USA). Confocal images were acquired as previously described (Xiao et al., J Biol Chem 288, 25297-25308 (2013); Xiao et al., J Clin Invest 123, 2207-2217 (2013)).

Quantification and statistics: For in vivo experiments, ten mice were used for each group. The sample size was determined according to the published literature. All data were statistically analyzed by one-way ANOVA with a Bonferroni correction, followed by Fisher's Exact Test. χ-squared test with 1 degree of freedom was applied to compare observed and estimated data. All error bars represent S.D. (standard deviation). Significance was presented as * when p<0.05, and ** when p<0.01. No significance was presented as NS. P value and n value were indicated in the figure legends.

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. A method of treating type 1 diabetes in a subject, comprising
administering to the subject a vector comprising a macrophage specific promoter operably linked to a nucleic acid molecule encoding TNF-alpha-induced protein 8-like 2 (TIPE2) protein,
wherein the vector is administered locally to a pancreas of the subject,
thereby polarizing macrophages to become M2 macrophages and treating the type 1 diabetes in the subject.

2. A method of polarizing macrophages to become M2 macrophages in the pancreas of a subject, comprising
administering to the subject a vector comprising a macrophage specific promoter operably linked to a nucleic acid molecule encoding TNF-alpha-induced protein 8-like 2 (TIPE2),
wherein the vector is administered locally to an organ of the subject,
thereby polarizing macrophages to become M2 macrophages in the pancreas of the subject.

3. The method of claim 2, wherein the organ is the pancreas.

4. The method of claim 3, wherein the subject has diabetes.

5. The method of claim 1, wherein the vector is administered intraductally into a pancreatic duct of the pancreas.

6. The method of claim 5, wherein intraductally administering comprises the use of endoscopic retrograde cholangiopancreatography (ERCP).

7. The method of any one of claim 1, wherein the vector is an adenovirus vector or an adeno-associated virus (AAV) vector.

8. The method of claim 7, wherein the vector is the AAV vector, and wherein the AAV vector is an AAV6 vector.

9. The method of claim 1, wherein the macrophage specific promoter is a CD11b promoter or a CD68 promoter.

10. The method of claim 9, wherein the macrophage specific promoter is the CD68 promoter.

11. The method of claim 1, wherein the TIPE2 protein comprises an amino acid sequence at least 95% identical to the amino acid sequence of SEQ ID NO: 1 or SEQ ID NO: 2.

12. The method of claim 11, wherein the TIPE2 protein comprises the amino acid sequence of SEQ ID NO: 1 or SEQ ID NO: 2.

13. The method of claim 1, wherein the subject is human.

14. The method of claim 1, wherein the method comprises administering an additional agent to the subject.

15. The method of claim 14, wherein the agent is an adenoviral or AAV vector encoding heterologous Pancreas duodenal homeobox protein (Pdx) 1 and Musculoaponeurotic fibrosarcoma oncogene homolog A (MafA).

16. (canceled)

17. A composition comprising:
a) a vector comprising a macrophage specific promoter operably linked to a nucleic acid molecule encoding TNF-alpha-induced protein 8-like 2 (TIPE2) protein;
b) a buffer; and
c) a contrast dye for endoscopic retrograde cholangiopancreatography.

18. The composition of claim 17, wherein the vector is an adenovirus vector or an adeno-associated virus (AAV) vector.

19. The composition of claim 18, wherein the vector is the AAV vector, and wherein the AAV vector is an AAV6 vector.

20. The composition of claim 17, wherein the macrophage specific promoter is a CD11b promoter or a CD68 promoter.

21. The composition of claim 20, wherein the macrophage specific promoter is the CD68 promoter.

22. The composition of claim 17, wherein the TIPE2 protein comprises an amino acid sequence at least 95% identical to the amino acid sequence of SEQ ID NO: 1 or SEQ ID NO: 2.

23. The composition of claim 22, wherein the TIPE2 protein comprises the amino acid sequence of SEQ ID NO: 1 or SEQ ID NO: 2.

24. (canceled)


SEQUENCE LISTING (6 sequences)

SEQ ID NO: 1 - protein, 184 aa, Homo sapiens
MESFSSKSLALQAEKKLLSKMAGRSVAHLFIDETSSEVLDELYRVSKEYTHSRPQAQRVIKDLIKVAIKVAVLHRNGSFGPSELALATRFRQKLRQGAMTALSFGEVDFTFEAAVLAGLLTECRDVLLELVEHHLTPKSHGRIRHVFDHFSDPGLLTALYGPDFTQHLGKICDGLRKLLDEGKL

SEQ ID NO: 2 - protein, 184 aa, Mus musculus
MESFSSKSLALQAEKKLLSKMAGRSVAHLFIDETSSEVLDELYRVSKEYTHSRPKAQRVIKDLIKVAVKVAVLHRSGCFGPGELALATRFRQKLRQGAMTALSFGEVDFTFEAAVLAGLLVECRDILLELVEHHLTPKSHDRIRHVFDHYSDPDLLAALYGPDFTQHLGKICDGLRKLLDEGKL

SEQ ID NO: 3 - DNA, 555 bp, Homo sapiens
atggagtccttcagctcaaagagcctggcactgcaagcagagaagaagctactgagtaagatggcgggtcgctctgtggctcatctcttcatagatgagacaagcagtgaggtgctagatgagctctaccgtgtgtccaaggagtacacgcacagccggccccaggcccagcgcgtgatcaaggacctgatcaaagtggccatcaaggtggctgtgctgcaccgcaatggctcctttggccccagtgagctggccctggctacccgctttcgccagaagctgcggcagggtgccatgacggcacttagctttggtgaggtagacttcaccttcgaggctgctgttctggctggcctgctgaccgagtgccgggatgtgctgctagagttggtggaacaccacctcacgcccaagtcacatggccgcatccgccacgtgtttgatcacttctctgacccaggtctgctcacggccctctatgggcctgacttcactcagcaccttggcaagatctgtgacggactcaggaagctgctagacgaagggaagctctga

SEQ ID NO: 4 - DNA, 555 bp, Mus musculus
atggagtccttcagctcaaagagtctggcactacaagcggagaagaagctgctgagtaaaatggctggtcggtccgtggcgcatctctttatcgacgagaccagcagcgaggtgctagacgagctttaccgcgtgtccaaagaatacacgcacagccggcccaaggcacagcgggtgatcaaagacctcatcaaggtagcggttaaagtggctgtgctgcaccgcagtggctgctttggccctggggagctggctctggctacacgatttcgtcagaagctacggcagggcgccatgaccgcacttagcttcggtgaggtggacttcacctttgaggctgccgtgctagcaggtctgctcgtcgagtgccgggacattctgctggagctggtggagcaccacctcacacccaagtcacatgaccgcatcaggcacgtgtttgatcactactctgaccccgacctgctggctgccctctatgggcctgacttcactcagcaccttggcaagatctgtgatgggctccggaagctgctggacgagggcaagctctga

SEQ ID NO: 5 - DNA, 5424 bp, Homo sapiens
gacggatcgggagatcctagcgtttaaacttaagcttggtaccgagctcggatccactagtccagtgtggtggaattctgcagatatcaaactgcctgtttgggcttctcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagcttacttgccacaactgccaagcaagtttcgctgagtttgacacatggatccctgtggatcaactgccctaggactccgtttgcacccatgtgacactgttgactttgccctgacgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagccatatcgaattctgctggggctactggcaggtaaggaggaaggaggctgaggggagggggcccctgggagggagcctgccctgggttgctaaccatctcctctctgccaaaagcccaggggactcagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagcca

SEQ ID NO: 6 - DNA, 4982 bp, Homo sapiens
agcttgcatgcctgcaggtcgactctagagtcgacctgcaggcatgcaagtttttttttttttttagagataagagtcttgctctgtcgcctaggctggagtgcagtggcacaatctctgctcactgcaacctccgcctccagggttcaagtgattctgctgcctcagcctcccaggtgggattacaggtgcctgccaccacgcctggctaatttttttgtctttttagtaaagatgaggtttcaccatgttgggcaggctggtttcaattgctgacctcaagtgagccaccccgcctcagcctcccaaaatgctaggattacaggcatgagccaccgcacccagccaagtttgtacatatatttttgactacacttcttaactattcttaggataaattactagaagtgaaaattcttgggtgaagagcttgaggcctttacacacacacacacacacacacacacacacacacaaataggctggatgcagtggctcacacctgtaatctcagcagtttgggaggctgaggaaggaggatcacttgagtccaggaggttgagaatagcctgaacaacatagcaagatcttgtctctacaaaaaatttaaaaaaaattagctggccatggcagcatgtgcctgtagtaccagctactcggaaggctgaggtaggaggatcgcttgagcccaggaggttgattgaagctgcagtgagctgtgattacaccactgcactccagcctgggcaacagagctagactctgtctctaaaaaaagcacaaaataatatttaaaaagcaccaggtatgcctgtacttgagttgtctttgttgatggctacaaatgaggacagctctggctgaagggcgcttccatttccatgggctgaaggagggacattttgcaaagtgtgttttcaggaagacacagagttttacctcctacacttgtttgatctgtattaatgtttgcttatttatttatttaattttttttttgagacagagtctcactctgtcacctgggctggagtgcagtggcattattgaggctcattgcagtctcagactcctgagctcaaacaatcctcctgcctcagcctctggagtagctaggactacaggcatgtgccaccatgcctggctaattttttaaatgtatttttttgtagagtcggggtctccctatgttgcccaggctggagtgcagtggtgtgatcctagctcactgcagcctggacctcgggctcaagtaattctcacacctcagcctgtccagtagcaggggctacaggcgcgcaccaccatgcccagctaattaaaaatatttttttgtagagacagggtctctctatgttgcccaggctggtttcaaactcccaggctcaagcaatcctcctgccttggcctcccaaagtgctggcattacaggcgtgagccactgcgcctggcccgtattaatgtttagaacacgaattccaggaggcaggctaagtctattcagcttgttcatatgcttgggccaacccaagaaacaagtgggtgacaaatggcaccttttggatagtggtattgactttgaaagtttgggtcaggaagctggggaggaagggtgggcaggctgtgggcagtcctgggcggaagaccaggcagggctatgtgctcactgagcctccgccctcttcctttgaatctctgatagacttctgcctcctacttctccttttctgcccttctttgctttggtggcttccttgtggttcctcagtggtgcctgcaacccctggttcacctccttccaggttctggctccttccagcccgggtaccgagctcgaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactca